(19) United States
(12) Patent Application Publication
(10) Pub. No.: US 2026/0328780 A1
(43) Pub. Date: Oct. 22, 2026

(54) BLOOD BIOMARKERS TO PREDICT RISK OF OSTEOARTHRITIS

(71) Applicant: Duke University, Durham, NC (US)

(72) Inventors: Virginia B. KRAUS, Durham, NC (US); Yi-Ju LI, Durham, NC (US)

(21) Appl. No.: 19/647,659

(22) Filed: Apr. 14, 2026

Related U.S. Application Data
(60) Provisional application No. 63/788,406, filed on Apr. 14, 2025.

(51) Int. Cl. G01N 33/68; G16H 50/20

(52) U.S. Cl. CPC G01N 33/6893; G16H 50/20; G01N 2800/105; G01N 2800/50; G01N 2800/52

(57) ABSTRACT

Provided herein are methods and biomarkers useful for predicting osteoarthritis in subjects in advance of any radiographic evidence. The biomarkers may also be used to develop treatment plans for subjects. Such treatment plans may help reduce symptoms or delay the progression of osteoarthritis.

### CROSS-REFERENCE TO RELATED APPLICATIONS

This application claims priority to U.S. Provisional Application No. 63/788,406 filed on Apr. 14, 2025, the contents of which is incorporated by reference in its entirety.

### STATEMENT REGARDING FEDERALLY SPONSORED RESEARCH

This invention was made with government support under AR071450 awarded by the National Institutes of Health. The government has certain rights in the invention.

### REFERENCE TO AN ELECTRONIC SEQUENCE LISTING

The contents of the electronic sequence listing (15555400814.xml; Size: 24,978 bytes; and Date of Creation: Apr. 10, 2026) is herein incorporated by reference in its entirety.

### BACKGROUND

Osteoarthritis (OA) is a prevalent chronic disease that represents a large and growing global health burden of large unmet need with respect to diagnostics, prognostics and therapeutics. Based on data from the Global Burden of Disease 2010 Study, musculoskeletal conditions are the second greatest cause of disability, as measured by years lived with disability (YLDs) worldwide and across most regions of the world; the main contributors are low back pain (83.1 million YLDs), neck pain (33.6 million YLDs) and osteoarthritis (17.1 million YLDs) with osteoarthritis of the knee accounting for 83% of this total. Globally, osteoarthritis of the knee affects 251 million individuals, and back and neck pain (likely largely also attributable to osteoarthritis) currently affect 964 million people worldwide. In the US, according to the Centers for Disease Control, osteoarthritis affects an estimated 26.9 million US adults (estimates from 2005, up 28% from the estimated 21 million US adults impacted in 1990). As the US population continues to age and struggle with obesity, the incidence and prevalence of the disease is expected to continue to grow. Consequently, the annual cost of osteoarthritis to the US, estimated to be $89.1 billion in 2001, is anticipated to continue to grow.

Demographics and baseline characteristics are poor predictors of OA progression including age, sex, body mass index, knee pain, general bone mineral content, and joint space width at baseline. In a systematic literature review, it was noted that 25-75% of painful knees cannot be diagnosed as OA by x-ray. Bedson J and Croft P R, BMC Musculoskelet Disord 9:116 (2008). Moreover, knee pain has been reported to have only a 23% sensitivity and 88% specificity for the diagnosis of radiographic OA. Hart et al., Ann Rheum Dis 50(7):467-70 (1991). Bedson et al concluded that, “The results of knee x-rays should not be used in isolation when assessing individual patients with knee pain.” Bedson J and Croft P R, BMC Musculoskelet Disord 9:116 (2008). Given the growing OA population globally and magnitude of the economic and societal burden, there is a growing interest in a new paradigm-switching from palliation, the traditional focus in OA, to early prevention. Accordingly, there is a remaining need in the art for better means of diagnosing and predicting osteoarthritis.

### SUMMARY

The present invention uses serum protein biomarkers to predict radiographic knee osteoarthritis (RKOA) years in advance of development. One aspect of the present disclosure provides a method comprising (a) obtaining a biological sample from a subject and (b) measuring expression of a biomarker in the biological sample, wherein the biomarker comprises at least a portion of at least two biomarkers selected from the group consisting of CRAC1, COMP, ZPI, ACTG, A2AP, APOB, APOE, C4BPB, C1R, C1S, CG4B, CO5, FBLN1, FBLN3, FINC1, HPT, HEMO, HRG, HABP2, PLF4, PRG4, RET4, LYAM1, VTNC, CD14, and combinations thereof, and wherein the biological sample comprises synovial fluid, chondrocytes, synoviocytes, blood, serum or plasma.

Another aspect of the present disclosure provides a method of identifying a subject at risk of developing osteoarthritis, the method comprising: (a) obtaining a sample from the subject, wherein the sample comprises synovial fluid, chondrocytes, synoviocytes, blood, serum or plasma; (b) measuring the expression of a biomarker in the sample, wherein the biomarker comprises at least a portion of at least one of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof, (c) comparing the expression of the biomarker(s) in the sample in step (b) to an expression level of the biomarker in a control sample; (d) identifying the subject at risk of developing osteoarthritis when the expression of the biomarker(s) is increased compared to the control; and (e) administering a treatment to the subject for osteoarthritis based on the risk identified in step (d).

Another aspect of the present disclosure provides a method for treating osteoarthritis in a subject comprising: (a) obtaining a biological sample from a subject; (b) measuring expression of a biomarker in the biological sample, wherein the biomarker comprises at least a portion of a protein selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and ombinations thereof, (c) comparing the expression of the biomarker to a control; (d) classifying the subject for likelihood of generation of osteoarthritis; and (e) administering osteoarthritis treatment to the subject wherein the level of the biomarker indicates the subject is likely to develop osteoarthritis.

In some embodiments, the biomarkers are selected from from the group consisting of CRAC1, COMP, ZPI, ACTG, APOB, APOE, C1S, CO5, FBLN1, FBLN3, HPT, HRG, HABP2, PLF4, PRG4, RET4, LYAM1, VTNC, CD14, and combinations thereof. In some embodiments, the biomarkers are selected from from the group consisting of CRAC1, COMP, ZPI, ACTG, APOB, APOE, C1S, CO5, FBLN1, FBLN3, HRG, HABP2, PLF4, PRG4, RET4, LYAM1, CD14, and combinations thereof. In some embodiments, the biomarkers are selected from the group consisting of CRAC1, COMP, ZPI, APOE, CO5, FBLN3, HRG, HABP2, PLF4, RET4, CD14, and combinations thereof. In some embodiments, the biomarkers comprise CRAC1, COMP, and ZPI. In some embodiments, the biomarkers further comprise FBLN3, PLF4 and RET4. In some embodiments, the biomarkers comprise at least one of ACTG₍₉₆₋₁₃₎, A2AP₍₁₅₁₋₁₆₁₎, APOB₍₁₃₃₋₁₃₉₎, APOB₍₄₀₂₂₋₄₀₃₄₎, APOE₍₂₇₀₋₂₇₈₎, C4BPB₍₁₁₉₋₁₃₅₎, CRAC1₍₁₀₁₋₁₀₈₎, CRAC1₍₁₇₀₋₁₇₈₎, COMP₍₆₅₂₋₆₆₅₎, C1R₍₆₈₃₋₆₈₉₎, C1S₍₅₁₆₋₅₂₃₎, CO4B₍₃₀₅₋₃₁₆₎, CO5₍₅₀₉₋₅₁₅₎, FBLN1₍₃₈₆₋₃₉₅₎, FBLN3₍₃₆₃₋₃₇₆₎, FINC₍₂₄₀₂₋₂₄₁₇₎, HPT₍₂₁₆₋₂₂₇₎, HEMO₍₁₉₈₋₂₀₈₎, HRG₍₁₄₀₋₁₅₃₎, HRG₍₁₈₈₋₂₀₁₎, HABP2₍₁₈₆₋₂₀₃₎, PLF4₍₈₂₋₉₂₎, ZPI₍₄₃₈₋₄₄₄₎, PRG4₍₁₁₈₅₋₁₂₀₁₎, RET4₍₁₈₅₋₁₉₅₎, LYAM1₍₇₁₋₇₈₎, VTNC₍₃₃₁₋₃₃₇₎, CD14₍₉₄₋₁₁₁₎, and combinations thereof. In some embodiments, the biomarkers comprise at least one of SEQ ID NO: 1-28, and sequences with at least 90% identity to any one of SEQ ID NO: 1-28. In some embodiments, the biological sample is a serum sample. In some embodiments, the measuring the expression comprises mass spectroscopy or an affinity reagent-based detection system. In some embodiments, the affinity reagent-based detection system is an ELISA, LFA or Western blot system. In some embodiments, the subject is over 50 years old. In some embodiments, the subject is at risk for osteoarthritis. In some embodiments, the subject has had a knee injury. In some embodiments, the knee injury comprises an injury to a knee ligament, a knee bone or knee cartilage. In some embodiments, the ligament knee injury comprises an injury to at least one of the anterior cruciate ligament (ACL), posterior cruciate ligament (PCL), medial collateral ligament (MCL), and lateral collateral ligament (LCL). In some embodiments, the knee injury comprises an injury to the meniscus, the tibia, the patella, or femur. In some embodiments the method further comprises, administering a therapeutic agent to the subject with increased expression of any one of the biomarkers and/or genes encoding the biomarkers or combinations thereof. In some embodiments, the therapeutic agent comprises an analgesic, a non-steroidal anti-inflammatory drugs (NSAIDs), steroid injections, surgery, an anti-metabolic syndrome drug, miRNA-based therapeutic or combinations thereof. In some embodiments the method further comprises determining a risk of the subject developing knee osteoarthritis based on the measured expression of the at least two biomarkers. In some embodiments, the risk of the subject developing knee osteoarthritis comprises applying a machine learning algorithm to the measured expression of the at least two biomarkers. In some embodiments, wherein the subject does not exhibit radiographic abnormalities or signs of knee osteoarthritis at the time of obtaining the biological sample. In some embodiments the method further comprises developing a treatment plan for the subject based on the determination of risk of the subject developing osteoarthritis.

In some embodiments, the subject had surgery to repair the injury to the knee. In some embodiments, the control sample is from a subject non suspected of having osteoarthritis or a standardized reference sample. In some embodiments, the control sample is from the same subject at a different time. In some embodiments, the sample is obtained from a subject within one year of a surgery.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1 is a diagram of the study design. This case-control cohort included a total of 200 female participants, matched for age and BMI, and followed for 10 years with three time points of radiograph collection and assessment for knee OA and two time points of blood sampling used for biomarkers analyses of this study. Hip radiographs were also obtained in y2. (Figure created with BioRender, agreement number WP266WMXQS).

FIG. 2 is a schematic of the serum peptide selection process.

FIG. 3 is a Venn diagram of “stable” proteins selected by elastic net regression. Indicated are the proteins corresponding to the stable set of peptide biomarkers predicting incident RKOA at y10 using serum biomarkers from y2, y6, or the y2/6-time integrated concentration (TIC); shared proteins appearing in more than one model are indicated by the overlap in the Venn diagram. (Figure created with BioRender, agreement number IR24YV6KXG).

FIG. 4 is a set of figures showing a backward elimination process to yield essential biomarker sets. (A) Y2 biomarkers and incidence of RKOA; (B) y6 biomarkers and incidence of RKOA; (C) y2/6-TIC (time integrated concentrations) biomarkers and incidence of RKOA. The biomarkers in the stable sets were sorted by the AUC drop associated with each single biomarker removal (from the left) from the model. Certain rules were applied to determine the stopping point (indicated by the arrow) as described in the methods. The AUC of each model was calculated as the average value of 10 imputed datasets. auc_boot, the average AUC when fitting on the bootstrapping samples; auc_orig, the average AUC when fitting on the original datasets; bias, the average difference between the original AUC and bootstrapping AUC.

FIG. 5 is a set of Forest plot and OR estimates for the multivariable models with the essential biomarkers. (A) Y2 biomarkers and incidence of knee OA (RKOA); (B) y6 biomarkers and incidence of knee RKOA; (C) y2/6-TIC (time integrated concentration) biomarkers and incidence of knee RKOA. Parameter estimates of each model were obtained by fitting on 10 imputed datasets. Rubin's rules were then applied to combine the estimates from the repeated complete data analyses. The results are presented as the ORs for an SD change of the corresponding peptides ratios.

FIG. 6 shows joint tissue gene expression profile corresponding to essential biomarkers. As previously described (21), single-cell RNA sequencing of medial tibial (MT) lesioned knee OA articular knee cartilage, outer lateral tibial (OLT) nonlesioned knee OA articular cartilage, and matched knee OA synovium (SY) were evaluated for number of cells expressing each of the essential biomarkers. Of 24 stable protein biomarkers, 18 were found in the transcriptome data and are plotted. Those highly expressed in both lesioned (medial tibial) and nonlesioned (outer lateral tibial) cartilage and synovium of OA joints included ACTG1, C1R, C1S, COMP, CRTAC1, EFEMP1 (FBLN3), and FN1. RBP4 (RET4) was highly expressed by chondrocytes from both lesioned and nonlesioned cartilage. FBLN1 and PRG4, although expressed in both chondrocytes and synoviocytes, were more strongly expressed in synoviocytes. A2AP, HABP2, HEMO (HPX), HRG, PLF4, and ZPI were not found in the joint tissue transcriptome data.

FIG. 7 shows STRING Network of non-overlapping year 2 and year 6 stable biomarker sets. Networks were generated for high confidence interactions (total interaction score >0.7) both without (on left) and with (on right) 20 interactors. Top) Year 2 biomarkers without and with interactors; Bottom) Year 6 biomarkers without and with biomarkers (Figure facilitated with BioRender, agreement number TN265XWSY4).

FIG. 8 shows networks generated by Ingenuity Pathways Analysis (IPA) for stable biomarker sets. Overlay analysis of the differentially expressed genes from the stable sets: A) Year 2 biomarkers and incidence of RKOA; B) Year 6 biomarkers and incidence of RKOA; C) Year 2/6-TIC biomarkers and incidence of RKOA. Shaded symbols represent genes that were upregulated in OA; the intensity of the shading indicates the degree of up-regulation. Open symbols represent nodes that are not shown in the stable biomarkers dataset but are included in the network based on previously known interactions. Different shapes represent different molecular classes. Solid lines represent direct interactions. Dashed lines represent indirect interactions. Arrows that circle up and over the symbols represent self-interaction. (Figure facilitated with BioRender, agreement number DZ2571C7BP).

### DETAILED DESCRIPTION

The present invention uses serum proteomic biomarkers to predict radiographic knee osteoarthritis (RKOA) years in advance of development in a case-control cohort of study participants selected for low risk of incident RKOA based on traditional risk factors. The inventors quantified serum biomarkers in sera (obtained years 2 and 6) that best predicted RKOA incidence at year 10. The ability of serum biomarkers to discriminate RKOA 4 to 8 years later in this cohort (AUCs, 0.76 to 0.77) was unexpected, it was also unexpected that these biomarkers were better at predicting the incidence of RKOA than magnetic resonance imaging, which was able to predict some features in other cohorts up to 1 to 5 years prior to RKOA (AUC ranges of 0.59 to 0.68).

The incident and progression biomarker panels described herein serve distinct but complementary clinical purposes along the osteoarthritis continuum. Biomarkers predicting incident OA identify individuals in a preradiographic molecular phase of disease, reflecting biological susceptibility to joint degeneration years before structural changes appear. In contrast, biomarkers predicting progression are applied to individuals with established OA to identify those at highest risk of rapid structural or symptomatic deterioration. The partial overlap between the panels reflects shared underlying pathophysiology rather than redundancy, consistent with the concept of a biological OA continuum. Importantly, identifying individuals in a preradiographic phase could enable lifestyle changes or preventive interventions such as targeted weight management, neuromuscular strengthening, injury prevention strategies, and enrollment in early-intervention trials. Several of the biomarkers identified in this study are also associated with progression of OA after radiographic diagnosis and these include ACTG, APOE, C1R, C1S, CG4B, CO5, CRAC1, HABP2, HEMO, HRG, PLF4, PRG4, RET4, and ZPI. Such biomarkers may be particularly valuable in younger high-risk populations, including athletes, individuals with prior knee injury or heavy joint loading such as military populations, where early identification could allow intervention during a stage when joint tissue pathologies remain modifiable, well before structural damage occurs.

### Methods

One aspect of the present disclosure provides a method comprising (a) obtaining a biological sample from a subject and (b) measuring expression of a biomarker in the biological sample, wherein the biomarker comprises at least a portion of at least two biomarkers selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14, and combinations thereof, and wherein the biological sample comprises synovial fluid, chondrocytes, synoviocytes, blood, serum or plasma.

Another aspect of the present disclosure comprises a method of identifying a subject at risk of developing osteoarthritis. In some embodiments, the method comprises (a) obtaining a sample from the subject, wherein the sample comprises synovial fluid, chondrocytes, synoviocytes, blood, serum or plasma; (b) measuring the expression of a biomarker in the sample, wherein the biomarker comprises at least a portion of at least one of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof, (c) comparing the expression of the biomarker(s) in the sample in step (b) to an expression level of the biomarker in a control sample; (d) identifying the subject at risk of developing osteoarthritis when the expression of the biomarker(s) is increased compared to the control; and (e) administering a treatment to the subject for osteoarthritis based on the risk identified in step (d).

Another aspect of the present disclosure provides a method for treating osteoarthritis in a subject comprising (a) obtaining a biological sample from a subject; (b) measuring expression of a biomarker in the biological sample, wherein the biomarker comprises at least a portion of a protein selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof, (c) comparing the expression of the biomarker to a control; (d) classifying the subject for likelihood of generation of osteoarthritis; and (e) administering osteoarthritis treatment to the subject wherein the level of the biomarker indicates the subject is likely to develop osteoarthritis.

As used herein, “biological sample” also known as a “sample”, “biological specimen” or “biological material”, is any material derived from a living organism or subject that can be studied in a laboratory or analyzed for diagnosing, predicting or treating a subject. Biological samples can be collected for purposes of diagnosing, predicting or treating a subject or as leftover materials from medical procedures. A biological sample may comprise any material derived from a subject, including, without limitation, blood, urine, tissue, cells, saliva, DNA, RNA, hair biopsy, serum, plasma, synovial fluid, or other biological fluid. In some embodiments, a biological sample as used herein may comprise a sample taken from a local site of arthritis, of suspected arthritis, or of an injury that increases the risk for arthritis. For example, synovial fluid, chondrocytes, synoviocytes from a subject having, suspected of having arthritis or having surgery on a potentially arthritic site. In some embodiments, a biological sample is a serum sample.

In some embodiments, a method comprising obtaining a biological sample from a subject and measuring the expression of a biomarker in the biological sample is provided. The biomarker may comprises a whole protein, or a peptide fragment of the protein. The biomarker may also comprise a gene encoding the protein or peptide. The biomarker may comprise a peptide or whole protein of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14, and combinations thereof. The biomarker may comprise a gene encoding a peptide or protein including CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14, and combinations thereof. In some embodiments, the biomarker is selected from those provided in Table 4. The peptides in Table 4 are SEQ ID NO: 1-28.

In some embodiments, the biomarker may be selected from the group consisting of CRAC1, COMP, ZPI, ACTG, APOB, APOE, C1S, CO5, FBLN1, FBLN3, HPT, HRG, HABP2, PLF4, PRG4, RET4, LYAM1, VTNC, CD14, and combinations thereof. In some embodiments, the biomarker may be selected from the group consisting of CRAC1, COMP, ZPI, ACTG, APOB, APOE, C1S, CO5, FBLN1, FBLN3, HRG, HABP2, PLF4, PRG4, RET4, LYAM1, CD14, and combinations thereof. In some embodiments, the biomarker may be selected from the group consisting of CRAC1, COMP, ZPI, APOE, CO5, FBLN3, HRG, HABP2, PLF4, RET4, CD14, and combinations thereof. In some embodiments the biomarker may comprise CRAC1, COMP, and ZPI or CRAC1, COMP, ZPI, FBLN3, PLF4 and RET4.

In some embodiments, the biomarker may comprise at least one of the peptides of ACTG₍₉₆₋₁₁₃₎, A2AP₍₁₅₁₋₁₆₁₎, APOB₍₁₃₃₋₁₃₉₎, APOB₍₄₀₂₂₋₄₀₃₄₎, APOE₍₂₇₀₋₂₇₈₎, C4BPB₍₁₁₉₋₁₃₅₎, CRAC1₍₁₀₁₋₁₀₈₎, CRAC1₍₁₇₀₋₁₇₈₎, COMP₍₆₅₂₋₆₆₅₎, C1R₍₆₈₃₋₆₈₉₎, C1S₍₅₁₆₋₅₂₃₎, CO4B₍₃₀₅₋₃₁₆₎, CO5₍₅₀₅₋₅₁₅₎, FBLN1₍₃₈₆₋₃₉₅₎, FBLN3₍₃₆₃₋₃₇₆₎, FINC₍₂₄₀₂₋₂₄₁₇₎, HPT₍₂₁₆₋₂₂₇₎, HEMO₍₁₉₈₋₂₀₈₎, HRG₍₁₄₀₋₅₃₎, HRG₍₁₈₈₋₂₀₁₎, HABP2₍₁₈₆₋₂₀₃₎, PLF4₍₈₂₋₉₂₎, ZPI₍₄₃₈₋₄₄₄₎, PRG4₍₁₁₈₅₋₁₂₀₁₎, RET4₍₁₈₅₋₁₉₅₎, LYAM1₍₇₁₋₇₈₎, VTNC₍₃₃₁₋₃₃₇₎, CD14₍₉₄₋₁₁₁₎ and combinations thereof. In some embodiments, the biomarker may comprise at least one peptide from SEQ ID NO: 1-28.

In some embodiments, the biomarkers provided herein may be combined with additional biomarkers. For example, those disclosed in U.S. Pat. No. 11,560,594, PCT publication WO2025/081155, which are incorporated by reference in their entirety herein. The present disclosure provides novel combinations of biomarkers and demonstrates unexpectedly that some of these biomarkers can be measured multiple years prior to radiographic evidence of arthritis, allowing the prediction of arthritis years in advance.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least four, at least 5, at least 6, at least 7, at least 8, at least 9, at least 10, at least 11, or at least 12 amino acids long. A protein, peptide, or polypeptide may refer to an individual protein or a collection of proteins. A peptide can be a portion of a larger protein, for example in Table 4 below, which demonstrates a protein, with multiple peptide sequences.

Accordingly, the term “gene”, refers to a polynucleotide that codes for a particular sequence of amino acids, which comprise all or part of one or more proteins.

As used herein, a biomarker is a measurable indicator of some biological state or condition. A biomarker is a characteristic that is objectively measured and evaluated as an indicator of normal biological processes, pathogenic processes, or pharmacologic responses to a therapeutic intervention. The biomarker can be a molecular biomarker, a cellular biomarker, a digital biomarker, a behavioral biomarker, or an imaging biomarker. The biomarker used herein may be evaluated from a biological sample such as blood, serum, plasma, synovial fluid, soft tissue, tissue biopsy or sample or urine. As used herein, a biomarker may be an indicator of arthritis or specifically of OA or an indicator of the severity or the prognosis for a subject with the disease or risk of developing the disease. In the present disclosure, a biomarker may be used to identify subjects that may benefit from a treatment disclosed herein, or may identify subjects to be diagnosed with osteoarthritis, or may provide a prognosis of arthritis in a subject. In some embodiments, the biomarkers provided herein may identify subjects 8 years, 7 years, 6 years, 5 years, 4 years, 3 years, 2 years, 1 year, and any time in-between prior to radiographic abnormality in the joint or in advance of potentially developing osteoarthritis.

Fragments and genetic variants of biomarkers are also encompassed by the present invention. “Fragment” is intended to include a portion of the amino acid sequence and hence a portion of the protein encoded thereby. A fragment or a biomarker peptide will generally encode at least 7, 8, 9, 10, 12, 15, 17, 20, 22, 25, 30 or more contiguous amino acids, or up to the total number of amino acids present in a full-length biomarker. Fragments of full-length proteins are provided as SEQ ID NO: 1-28 and in Table 4. “Variant” is intended to mean substantially similar sequences. Generally, variants of a particular biomarker of the invention will have at least about 80%, 85%, 90%, 91%, 92%, 93%, 94%, 95%, 96%, 97%, 98%, 99% or more amino acid identity to that biomarker as determined by amino acid alignment programs. Variants may simply be exemplified as allelic variants of a protein present in a population of subjects.

Modified forms of the biomarkers or pathogenic peptides are also encompassed by the present invention. Modifications may include post-translational modifications including, but not limited to phosphorylation, glycosylation, ubiquitination, methylation, deamination, hydroxylation, lipidation, acetylation, SUMOylation, or proteolysis.

Any methods available in the art for detecting the level of biomarkers are encompassed herein. The level of a biomarker of the invention can be detected using a peptide corresponding to the biomarker. “Measuring an expression level of” is intended to mean determining the quantity or presence of a biomarker (i.e., peptide) in a sample for at least one of the biomarkers of Table 4, or any of the sequences provided herein. Thus, “measuring an expression level of” encompasses instances where a biomarker is determined not to be detectable due to failure to be produced, or due to production below the detection limit of the assay; “measuring an expression level of” also encompasses low, normal and high levels of detection. Measuring an expression level also includes instances where a marker is degraded or is more stable in a person with osteoarthritis or with progressive disease and is not limited to production of new peptide or the timing of peptide production.

Methods suitable for “measuring an expression level of” biomarkers are known to those of skill in the art and include, but are not limited to, ELISA, immunofluorescence, flow cytometry analysis, Western blot, magnetic immunoassays, mass spectrometry, gel electrophoresis, antibody-based microarrays and non-antibody-based microarrays or combinations of these methods. In the Examples, LC-MRM (liquid chromatography-multiple reaction monitoring mass spectroscopy) was used. In some embodiments, the biomarker is measured using an antibody or other affinity reagent-based capture method. In some embodiments, the affinity reagent-based detection system is an ELISA, LFA or Western blot system. In some embodiments, the biomarker is measured using mass spectrometry. In some embodiments, the expression of the biomarker may be measured, for example the gene or protein expression.

The level of the biomarker in the sample from the subject may be quantified directly or compared to a reference or control level of the biomarker. For a biomarker that is directly quantified, the presence or absence of a biomarker may be used. Alternatively, the expression of the biomarker may be evaluated relative to a control or reference. The reference level may be determined empirically such as illustrated in the Examples, by comparison to the levels found in a set of samples from subjects with known clinical outcomes or known to have or not have osteoarthritis. Alternatively, the reference level may be a level of the biomarker found in samples, such as serum samples, which becomes a standard and can be used as a predictor for new samples. The level of the biomarker in the sample from the subject may be increased or decreased (i.e., “altered”) as compared to the reference level. The Example provided herein provides information regarding how each biomarker is altered to indicate a diagnosis or to predict progression. Some biomarkers were positively associated with OR (odd ratios) ranges between 1.36-4.09 and include ACTG, APOB, APOE, COMP, CRAC1, HABP2, HPT, HRG, and ZPI. Other biomarkers were negatively associated with OR ranges 0.35-0.64 and include C1S, CO5, FBLN1, FBLN3, LYAM1, PLF4, PRG4, RET4, and VTNC. In some embodiments, the control is a sample from a subject not having arthritis, or not suspected of having arthritis, or not having risk factors for arthritis. In some embodiments, the control is from the same subject at a different time. For example, at an earlier age, before starting a therapeutic, before or after surgery or before or after an injury.

As used herein, “subject” or “patient” refers to both mammals and non-mammals. “Mammals” include any member of the class Mammalia, such as humans, non-human primates (e.g., chimpanzees, other apes and monkey species), farm animals (e.g., cattle, horses, sheep, goats, and swine), domestic animals (e.g., rabbits, dogs, and cats), and laboratory animals (e.g., rats, mice, and guinea pigs). The term “subject” does not denote a particular age or sex. In the Examples women were used as the subjects, but the results are expected to apply equally to men. In one embodiment, the subject is a human. A “subject” or “subject in need thereof” as utilized herein may refer to a subject in need of treatment for a disease or disorder associated with arthritis, including osteoarthritis or a condition that increases the risk of arthritis, or may be diagnosed with arthritis.

In some embodiments, the subject is a human at a normal health and wellness screening, or a subject who is aging, who has experienced a joint injury, who experiences professional or lifestyle risks associated with overuse or injury to the knees or a person otherwise at risk of arthritis. A subject “at risk of arthritis” refers to an individual who does not yet exhibit clinically confirmed arthritis, but possesses one or more physiological, genetic, environmental, or clinical characteristics that increase the likelihood of developing arthritis compared to the general population. In certain embodiments, a subject at risk includes, but is not limited to, an individual who meets one or more of the following criteria: family history indicators, genetic indicators, clinical or physiological indicators, environmental or lifestyle factors, and age-related or metabolic risk factors. Family history indicators may include, a first-degree or second-degree relative diagnosed with rheumatoid arthritis, osteoarthritis, psoriatic arthritis, gout, juvenile idiopathic arthritis, or another inflammatory arthropathy. Environmental or lifestyle factors may include exposure to environmental or infectious triggers associated with increased arthritis risk and occupations with heavy joint loading, such as military personnel, or regularly participating in activities with heavy joint loading. Age-related or metabolic risk may include subjects of advanced age, or subjects with metabolic disorders such as diabetes, metabolic syndrome, or hyperuricemia known to predispose to arthritic disease. In some embodiments, the subject is over 50 years of age, for example 52, 55, 60, 65, 70, 75, 80, 85 or more in age.

Clinical or physiological indicators may include, but are not limited to, a history of joint pain, stiffness, or swelling without meeting diagnostic criteria for arthritis, presence of autoimmune or inflammatory conditions known to predispose to arthritis, such as psoriasis, inflammatory bowel disease, or lupus, evidence of early joint degeneration on imaging (e.g., MRI, ultrasound) in the absence of full clinical disease. Clinical or physiological indicators may also include subjects with joint, bone ligament or tendon injuries. In some embodiments, the subject has had a knee injury, including an injury to a knee ligament, a knee bone or knee cartilage. In some embodiments, the ligament knee injury comprises an injury to at least one of the anterior cruciate ligament (ACL), posterior cruciate ligament (PCL), medial collateral ligament (MCL), and lateral collateral ligament (LCL). In some embodiments, the knee injury comprises an injury to the meniscus, the tibia, the patella, or femur. In some embodiments, the subject has had surgery to a joint, for example knee surgery to repair a ligament or a knee replacement surgery. In some embodiments, a biological sample may be obtained during a surgery to repair a knee injury. In some embodiments, the sample is obtained from a subject within one year of a surgery. In some embodiments, the biomarkers may be used as a screening tool for the condition of a subject's joints.

In some embodiments, the subject has an arthritis that increases the risk of OA. For example, a subject may have Rheumatoid arthritis, Psoriatic arthritis, or Gout, which all increase the risk of developing OA.

A subject may include a subject suspected of having or having arthritis that is characterized by gross abnormality visible by X-ray, computerized tomography (CT), or magnetic resonance imaging (MRI), joint symptoms, medical history or physical exam.

The predictive methods described herein may be combined to provide increased significance of the results, i.e. increased AUCs. For example, the levels of multiple markers may be determined in a sample from the subject and the results may have additional statistical or predictive power via the combination. The levels may be compared to the reference levels and a diagnosis or a prediction of risk of progression made. Several exemplary combinations are provided herein and in the Example, but any combination of 2, 3, 4, 5, 6, 7, 8, 9, 10 or more of the biomarkers may provide a prediction with increased accuracy and thus be beneficial. Several biomarker combinations achieved AUCs of 0.76-0.77 and combining biomarkers with clinical variables (age, BMI, WOMAC pain, hip OA) boosted AUCs to 0.83.

In some embodiments, the method further comprises administering a therapeutic agent to the subject with increased expression of any one of the biomarkers and/or genes encoding the biomarkers or combinations thereof. Without limitation, the therapeutic agent may comprise an analgesic, non-steroidal anti-inflammatory drugs (NSAIDs), steroid injections, surgery including joint repair or joint replacement, or combinations thereof. Suitable anti-inflammatory therapeutics are known to those skilled in the art and may include, without limitation, nonsteroidal anti-inflammatory drugs (NSAIDs), disease-modifying osteoarthritis drugs (DMOADs), disease-modifying antirheumatic drugs (DMARDs), corticosteroids, and hyaluronans. Several classes of DMARDs may be used in accordance with the present invention including, but not limited to, traditional DMARDs such as methotrexate, hydroxycholorquine, sulfasalazine, leflunomide, cyclophosphamide and azathioprine; biologics such as anti-IL-1 therapeutics, anti-TNF therapeutics, metalloproteinase inhibitors, p38 inhibitors, abatacept, adalimumab, anakinra, certolizumab pegol, etanercept, infliximab, golimumab and rituximab; and JAK inhibitors such as Tofacitinib. Suitable anti-pain therapeutics include, without limitation, non-opioid analgesics (e.g., acetaminophen), nonsteroidal anti-inflammatory drugs (NSAIDs), opioid analgesics, and co-analgesics and most likely in future, nerve growth factor inhibitors. In some embodiments, the therapeutic agent comprises an analgesic, a non-steroidal anti-inflammatory drugs (NSAIDs), steroid injections, surgery, an anti-metabolic syndrome drug, miRNA-based therapeutic or combinations thereof.

Therapeutic agents may also include those that treat associated risk factors for developing arthritis. For example, a therapeutic agent that treats metabolic disorders (i.e. an anti-metabolic syndrome drug) such as diabetes, metabolic syndrome, or hyperuricemia may be used with the methods described herein. Examples of such therapeutic agents include, but are not limited to anti-diabetic agents (e.g. insulin, biguanides, GLP-1 receptor agonists, DPP-4 inhibitors, thiazolidinediones etc.), anti-obesity drugs (e.g. Orlistat, Phentermine/topiramate, Naltrexone/bupropion, GLP-1 receptor agonists used at obesity doses etc.), lipid lowering agents (e.g. statins, PCSK9, Ezetimibe etc.), anti-hypertensive agents (e.g. ACE inhibitors, Angiotensin II receptor blockers, Calcium channel blockers etc.).

Therapeutic agents may also include polynucleotide-based therapeutics, including micro RNA (miRNA)-based therapeutics. In some embodiments, the miRNA may comprise those listed in WO 2026/073074 and Hsueh M F, Önnerfjord P, Kraus V B. Anabolic indices of matrix proteins identify regenerative small RNA intrinsic to human cartilage. Sci Adv. 2025 Jul. 11; 11(28):eadu8440. doi: 10.1126/sciadv.adu8440. Epub 2025 Jul. 11. PMID: 40644557. which are incorporated by reference in their entirety herein.

In some embodiments, the methods provided herein further comprise determining a risk of the subject developing knee osteoarthritis based on the measured expression of the at least two biomarkers. “Determining the risk” may include qualitative or quantitative analyses, including comparing obtained data to reference values or thresholds, applying predictive algorithms or models, or identifying the presence or absence of one or more biomarkers associated with increased probability of disease onset or progression. In some embodiments, determining the risk of the subject developing knee osteoarthritis comprises applying a machine learning algorithm to the measured expression of the at least two biomarkers. The machine learning algorithm may include approaches such as elastic-net logistic regression and bootstrapping strategy as described in the examples. In some embodiments, the subject does not exhibit radiographic abnormalities or signs of knee osteoarthritis at the time of obtaining the biological sample.

In some embodiments, the method further comprises developing a treatment plan for the subject based on the determination of risk of the subject developing osteoarthritis. As used herein, “developing a treatment plan” refers to the process of identifying, formulating, selecting, or modifying one or more therapeutic actions, interventions, or regimens for a subject based on clinical, biological, genetic, environmental, or other relevant information obtained from or about the subject, for example, based on the measurement of the biomarkers described herein. The treatment plan may include, but is not limited to, selecting a therapeutic agent, determining a dosing schedule, identifying lifestyle or behavioral interventions, recommending diagnostic follow-up, or adjusting existing therapies to address the subject's condition, risk profile, or predicted response.” In some embodiments, the treatment plan comprises additional measurements of the biomarkers provided herein. In some embodiments, developing a treatment plan comprises administering a treatment.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

The following Examples are illustrative and should not be interpreted to limit the scope of the claimed subject matter.

Reference is made to the manuscript: “Kraus V B, et al. An osteoarthritis pathophysiological continuum revealed by molecular biomarkers. Sci Adv. 2024 Apr. 26; 10(17):eadj6814. Epub 2024 Apr. 26. PMID: 38669329; PMCID: PMC11051665,” the content of which is incorporated herein by reference in its entirety.

### Example 1

There has been a 9% global increase in the age standardized osteoarthritis (OA) incidence rate over the 28-year period from 1990 to 2017 (1). Given the growing OA population globally and magnitude of the economic and societal burden (2), there is a growing interest in a new paradigm—switching from palliation, the traditional focus in OA, to early prevention. Early-stage OA could provide a “window of opportunity” in which to arrest the disease process and restore joint homeostasis (3). Efforts are ongoing to establish classification criteria for early OA (3-5). The current goal of classification criteria for early-stage knee OA is to discriminate those with joint symptoms at increased risk of OA structural progression, from those with symptoms due to other causes. However, OA has a long prodromal phase, possibly in part asymptomatic, most certainly preradiographic (6-8). The identification of sensitive biomarkers to identify early OA, before development of overt structural damage, has the potential to greatly reduce the medical burden of disease. This is posited because disease-modifying drugs, once available, are expected to have the greatest favorable impact on OA if administered early in the disease course before irreversible radiographic abnormalities (7). However, the majority of biomarker research has focused on established radiographic knee OA (RKOA) (9-11); the task of developing measures with utility in individual patients is arduous (12). There have been some molecular biomarkers identified to predict incident RKOA, but results are conflicting, for instance, for serum Cartilage Oligomeric Matrix Protein (COMP) [positively (13-17) and negatively (18) associated], and urinary C-terminal cross-linked telopeptides of type II collagen (CTXII) [positively (16, 17) and negatively (18) associated]. Therefore, the identification of robust biochemical predictors of RKOA incidence is a challenge and remains an unmet need.

By definition, all individuals who develop incident RKOA have had progression of a pathological process from a subclinical to a clinically detectable stage with joint symptoms, imaging abnormality, or both. We are interested in determining whether the molecular pathophysiology in the decade before incident RKOA bears any similarity to the molecular pathophysiology of progression of “established” RKOA. We hypothesize that molecular entities predicting incident RKOA are similar to those predicting progression of RKOA because of a similar underlying molecular pathophysiology, thereby supporting the concept of a biological OA continuum. To test this hypothesis, we evaluated a serum proteomic biomarker panel that we showed predicted RKOA progression with initial discovery in the Foundation for National Institutes of Health (FNIH) cohort [a subset of individuals from the Osteoarthritis Initiative (OAI) with established RKOA] and with validation in the Biomarker Factory cohort (19). The current study was designed to evaluate the proteomic panel for its ability to predict incident RKOA in the Chingford 1000 Women study, a longitudinal cohort of middle-aged women with RKOA data available over 10 years. In our prior study of 649 women in the Chingford study, we identified knee level radiographic factors, added to clinical factors, as important for prediction of incident RKOA (20). In the current study, we aimed to use serum proteomic biomarkers to predict incident RKOA in a case-control cohort of Chingford study participants selected for low risk of incident RKOA based on traditional risk factors [excluded prior joint injury and any baseline knee OA of Kellgren-Lawrence (KL) grade >1]. We quantified serum peptides using targeted multiple reaction monitoring (MRM) mass spectrometry. Machine learning techniques were used to select proteomic biomarkers in sera (obtained years 2 and 6) that best predicted RKOA incidence at year 10.

### Results

### Participant Characteristics

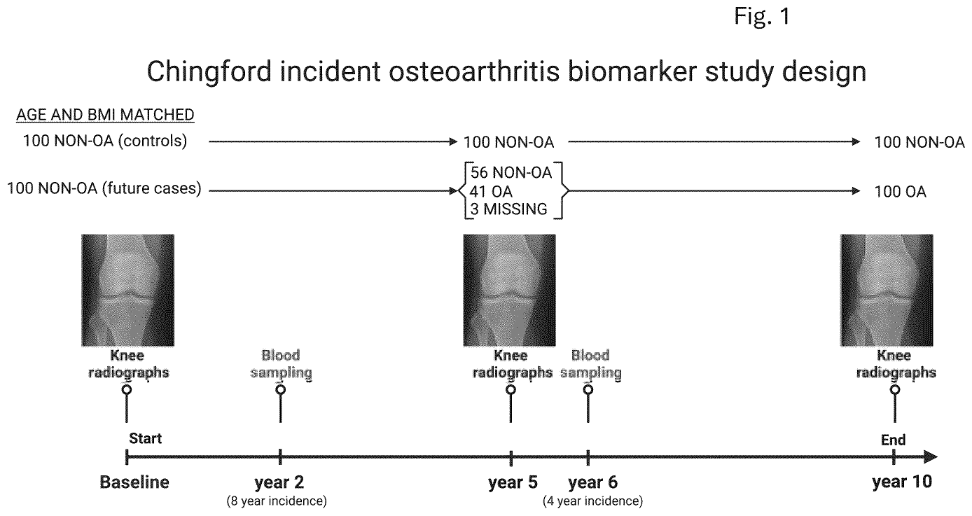

In total, 200 white female participants from the UK were studied (FIG. 1, study design), consisting of 100 incident RKOA cases and 100 age- and body mass index (BMI)-matched controls; their baseline characteristics included mean (SD): age 54 (6) years and BMI 26 (4) kg/m² (Table 1). As expected, because of the selection criteria, there were no statistically significant group differences based on baseline age or BMI. There were no discernible clinical differences related to baseline KL knee GA severity score 0 versus 1 (Table 3). Few participants (16%) reported any baseline pain on walking; however, excluding those with missing data, more incident cases reported baseline pain on walking compared with controls (P=0.03, Table 1). At baseline, 12 individuals (six controls and six RKOA) reported some use of an analgesic (majority ibuprofen); no individuals reported osteoporosis medication use, and only one control individual reported hormone replacement therapy use. The number of individuals with KL3/4 of at least one knee was 9 at year 5 (y5) and 23 at y10.

TABLE 1
Characteristics of study participants by knee radiographic OA
incidence status. RKOA, radiographic knee osteoarthritis.

| | | | RKOA | | | | |
|---|---|---|---|---|---|---|---|
| | | | incidence | | Total | | |
| | (N = 100) | | (N = 100) | | (N = 200) | | P value |
| Baseline age | | | | | | | 0.76* |
| Mean (Sd) | 54.16 | (5.46) | 54.42 | (5.93) | 54.29 | (5.68) | |
| Range | (45.00-65.00) | | (45.00-65.00) | | (45.00-65.00) | | |
| Smoking status | | | | | | | 0.33† |
| nonsmoker | 62 | (62%) | 52 | (52%) | 114 | (57%) | |
| current smoker | 15 | (15%) | 21 | (21%) | 36 | (18%) | |
| Past smoker | 23 | (23%) | 27 | (27%) | 50 | (25%) | |
| Year 1 BMI | | | | | | | 0.75* |
| Mean (Sd) | 25.79 | (3.57) | 25.92 | (4.48) | 25.86 | (4.04) | |
| Range | (16.84-36.00) | | (18.37-44.46) | | (16.84-44.46) | | |
| Baseline regular | | | | | | | 0.83† |
| activity | | | | | | | |
| Missing | 1 | (1%) | 2 | (2%) | 3 | (1.5%) | |
| no | 69 | (69%) | 67 | (67%) | 136 | (68%) | |
| Yes | 30 | (30%) | 31 | (31%) | 61 | (30.5%) | |
| Baseline pain | | | | | | | |
| walking | | | | | | | |
| (WOMAC A1) | | | | | | | |
| Missing | 15 | (15%) | 15 | (15%) | 30 | (15%) | |
| none | 75 | (75%) | 63 | (63%) | 138 | (69%) | Reference |
| Yes | 10 | (10%) | 22 | (22%) | 32 | (16%) | 0.03† |
| Year 2 hip OA | | | | | | | |
| (KL ≥ 2) | | | | | | | |
| Missing | 0 | (0%) | 2 | (2%) | 2 | (1%) | |
| none | 100 | (100%) | 82 | (82%) | 182 | (91%) | Reference |
| Yes | 0 | (0%) | 16 | (16%) | 16 | (8%) | 0.00008 |
| Baseline KL grade | | | | | | | 0.0003 |
| Kl0/0 | 98 | (98%) | 80 | (80%) | 178 | (89%) | |
| Kl1/0 or 0/1 | 1 | (1%) | 8 | (8%) | 9 | (4.5%) | |
| Kl1/1 | 1 | (1%) | 12 | (12%) | 13 | (6.5%) | |

*Wilcoxon was used for comparing the difference between the two groups excluding missings.
†Chi-square test was used for comparing the difference between the two groups excluding missings.

TABLE 3
Baseline characteristics of all study participants based on knee KL categories

| | Bilateral Knee Baseline KL grade categories | | | | | | | |
|---|---|---|---|---|---|---|---|---|
| | 0/0 | | 0/1 or 1/0 | | 1/1 | | Overall | |
| | (N = 178) | | (N = 9) | | (N = 13) | | (N = 200) | |
| Baseline age | | | | | | | | |
| Mean (SD) | 54.1 | (5.62) | 54.9 | (7.03) | 56.5 | (5.58) | 54.3 | (5.68) |
| Median [Min, Max] | 54.0 | [45.0, 65.0] | 54.0 | [47.0, 65.0] | 58.0 | [47.0, 63.0] | 54.0 | [45.0, 65.0] |
| Smoking status | | | | | | | | |
| nonsmoker | 102 | (57.3%) | 5 | (55.6%) | 7 | (53.8%) | 114 | (57.0%) |
| current smoker | 31 | (17.4%) | 2 | (22.2%) | 3 | (23.1%) | 36 | (18.0%) |
| past smoker | 45 | (25.3%) | 2 | (22.2%) | 3 | (23.1%) | 50 | (25.0%) |
| Year 1 BMI | | | | | | | | |
| Mean (SD) | 25.8 | (4.01) | 27.2 | (4.81) | | 25.4 (4.04) | 25.9 | (4.04) |
| Median [Min, Max] | 25.2 | [16.8, 44.5] | 25.3 | [23.3, 38.8] | 26.4 | [19.0, 31.6] | 25.2 | [16.8, 44.5] |
| Baseline regular | | | | | | | | |
| activity | | | | | | | | |
| No | 122 | (68.5%) | 6 | (66.7%) | 8 | (61.5%) | 136 | (68.0%) |
| Yes | 54 | (30.3%) | 3 | (33.3%) | 4 | (30.8%) | 61 | (30.5%) |
| Missing | 2 | (1.1%) | 0 | (0%) | 1 | (7.7%) | 3 | (1.5%) |
| WOMAC Pain | | | | | | | | |
| walking | | | | | | | | |
| (WOMAC A1) | | | | | | | | |
| None | 126 | (70.8%) | 6 | (66.7%) | 6 | (46.2%) | 138 | (69.0%) |
| Yes | 30 | (16.9%) | 0 | (0%) | 2 | (15.4%) | 32 | (16.0%) |
| Missing | 22 | (12.4%) | 3 | (33.3%) | 5 | (38.5%) | 30 | (15.0%) |
| Year 2 Hip OA | | | | | | | | |
| Yes | 13 | (7.3%) | 2 | (22.2%) | 1 | (7.7%) | 16 | (8.0%) |
| No | 163 | (91.6%) | 7 | (77.8%) | 12 | (92.3%) | 182 | (91.0%) |
| Missing | 2 | (1.1%) | 0 | (0%) | 0 | (0%) | 2 | (1.0%) |

### Stable Biomarkers

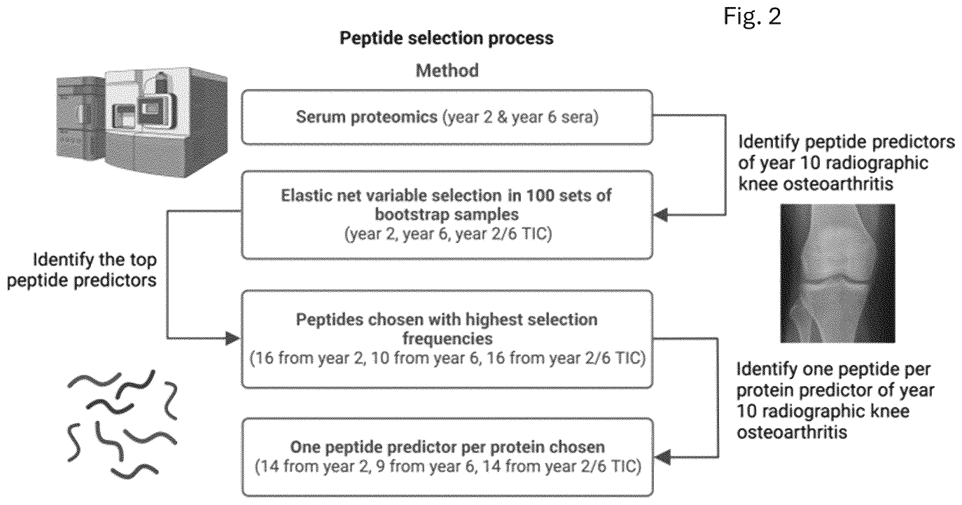

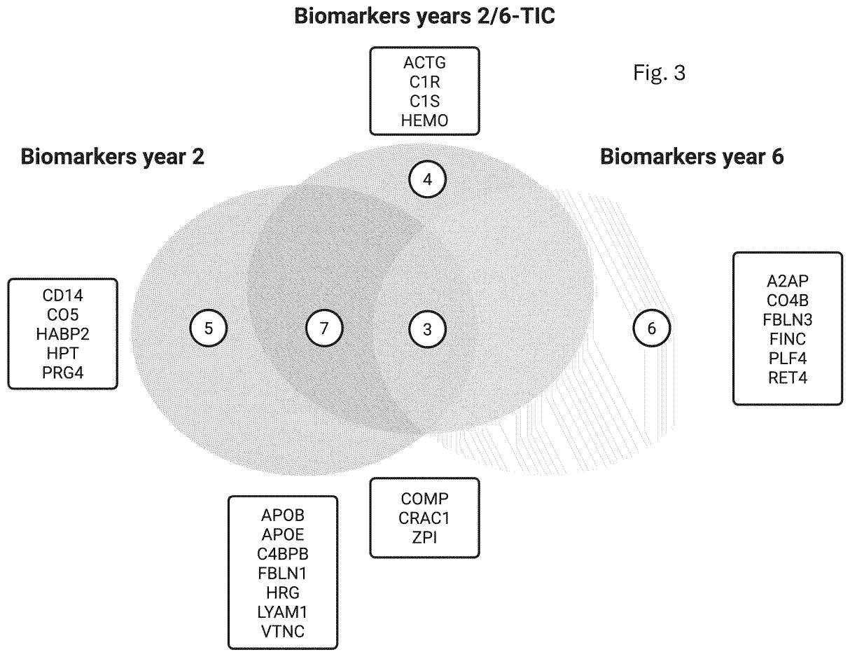

To identify serum peptides predicting incident RKOA, we used a validated, targeted, MRM assay consisting of 165 peptides (104 proteins) (19) to analyze nondepleted serum samples obtained from the 200 study participants in y2 and y6, totaling 400 samples (FIG. 2). A total of 115 peptides in 393 samples passed quality control (QC) measures for subsequent statistical analyses (196 from y2 and 197 from y6). A total of 16, 10, and 16 peptides were chosen most frequently by elastic net regression as predictors of incident RKOA at y10 based on serum biomarker concentrations from y2, y6, and y2/6-time-integrated concentrations (TICs), respectively (peptide amino acid designations provided in subscripts, listed in Table 4). After reducing the predictor sets to one representative peptide per protein, the final number of predictors was 14, 9, and 14 peptides at each time point; these were termed the “stable set” biomarkers (FIG. 3). In total, from the three analyses, 27 peptides (24 proteins) were identified as stable set predictors of incident RKOA. Three peptides, COMP₍₆₅₂₋₆₆₅₎, CRAC1₍₁₀₁₋₁₀₈₎, and ZPI₍₄₃₈₋₄₄₄₎, were selected as part of all three stable biomarker sets.

TABLE 4
Stable Set of Peptides measured in Chingford Cohort sera by targeted mass spectrometry
multiple reaction monitoring (MRM)

| | Gene | Peptide | SEQ ID | Amino Acid | Accession |
|---|---|---|---|---|---|
| Protein Name | Name | Sequence | NO: | Numbers** | Number |
| Actin, | ACTG1 | VAPEEHPVLLTE | 1 | 96-113 | P63261-1 |
| cytoplasmic 2 | | APLNPK | | | |
| (ACTG)* | | | | | |
| Alpha-2- | SERPINF2 | LCQDLGPGAFR | 2 | 151-161 | P08697-1 |
| antiplasmin | | | | | |
| (A2AP) | | | | | |
| Apolipoprotein | APOB | LAIPEGK | 3 | 133-139 | P04114-1 |
| B-100 | | | | | |
| (APOB) | | | | | |
| Apolipoprotein | APOB | WNFYYSPQSSPD | 4 | 4022-4034 | P04114-1 |
| B-100 | | K | | | |
| (APOB) | | | | | |
| Apolipoprotein | APOE | LQAEAFQAR | 5 | 270-278 | P02649-1 |
| E (APOE) | | | | | |
| C4b-binding | C4BPB | SQCLEDHTWAP | 6 | 119-135 | P20851-1 |
| protein beta | | PFPICK | | | |
| chain | | | | | |
| (C4BPB) | | | | | |
| Cartilage | CRTAC1 | SSPYYALR | 7 | 101-108 | Q9NQ79- |
| acidic protein | | | | | 1 |
| 1 (CRAC1) | | | | | |
| Cartilage | CRTAC1 | GVASLFAGR | 8 | 170-178 | Q9NQ79- |
| acidic protein | | | | | 1 |
| 1 (CRAC1) | | | | | |
| Cartilage | COMP | NALWHTGDTES | 9 | 652-665 | P49747-1 |
| oligomeric | | QVR | | | |
| matrix protein | | | | | |
| (COMP) | | | | | |
| Complement | C1R | GYGFYTK | 10 | 683-689 | P00736-1 |
| C1r | | | | | |
| subcomponent | | | | | |
| (C1R) | | | | | |
| Complement | C1S | LLEVPEGR | 11 | 516-523 | P09871-1 |
| C1s | | | | | |
| subcomponent | | | | | |
| (C1S) | | | | | |
| Complement | C4B | LVNGQSHISLSK | 12 | 305-316 | P0C0L5-1 |
| C4-B (CO4B) | | | | | |
| Complement | C5 | IIHFGTR | 13 | 509-515 | P01031-1 |
| C5 (CO5) | | | | | |
| Fibulin-1 | FBLN1 | TGYYFDGISR | 14 | 386-395 | P23142-1 |
| (FBLN1) | | | | | |
| EGF- | EFEMP1 | NPCQDPYILTPE | 15 | 363-376 | Q12805-1 |
| containing | | NR | | | |
| fibulin-like | | | | | |
| extracellular | | | | | |
| matrix protein | | | | | |
| 1 (FBLN3) | | | | | |
| Fibronectin | FN1 | EYLGAICSCTCF | 16 | 2402-2417 | P02751- |
| (FINC) | | GGQR | | | 15 |
| Haptoglobin | HP | DIAPTLTLYVGK | 17 | 216-227 | P00738-1 |
| (HPT) | | | | | |
| Hemopexin | HPX | YYCFQGNQFLR | 18 | 198-208 | P02790-1 |
| (HEMO) | | | | | |
| Histidine-rich | HRG | DSPVLIDFFEDTE | 19 | 140-153 | P04196-1 |
| glycoprotein | | R | | | |
| (HRG) | | | | | |
| Histidine-rich | HRG | GGEGTGYFVDFS | 20 | 188-201 | P04196-1 |
| glycoprotein | | VR | | | |
| (HRG) | | | | | |
| Hyaluronan- | HABP2 | FCEIGSDDCYVG | 21 | 186-203 | Q14520-1 |
| binding | | DGYSYR | | | |
| protein 2 | | | | | |
| (HABP2) | | | | | |
| Platelet factor | PF4 | ICLDLQAPLYK | 22 | 82-92 | P02776-1 |
| 4 (PLF4) | | | | | |
| Protein Z- | SERPINA10 | VVNPTLL | 23 | 438-444 | Q9UK55- |
| dependent | | | | | 1 |
| protease | | | | | |
| inhibitor (ZPI) | | | | | |
| Proteoglycan 4 | PRG4 | ITEVWGIPSPIDT | 24 | 1185-1201 | P51884-1 |
| (PRG4) | | VFTR | | | |
| Retinol- | RBP4 | LIVHNGYCDGR | 25 | 185-195 | P02753-1 |
| binding | | | | | |
| protein 4 | | | | | |
| RET4) | | | | | |
| L-Selectin | SELL | AEIEYLEK | 26 | 71-78 | P14151-1 |
| (LYAM1) | | | | | |
| Vitronectin | VTN | QPQFISR | 27 | 331-337 | P04004-1 |
| (VTNC) | | | | | |
| CD14 | CD14 | LTVGAAQVPAQ | 28 | 94-111 | P08571 |
| | | LLVGALR | | | |

### Essential Biomarkers and Model Performance for Predicting Incident RKOA

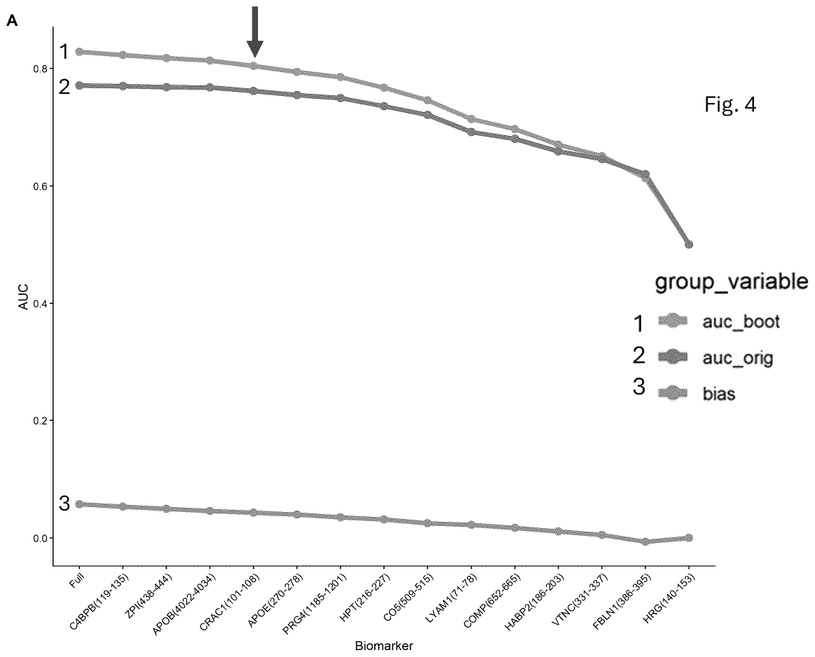

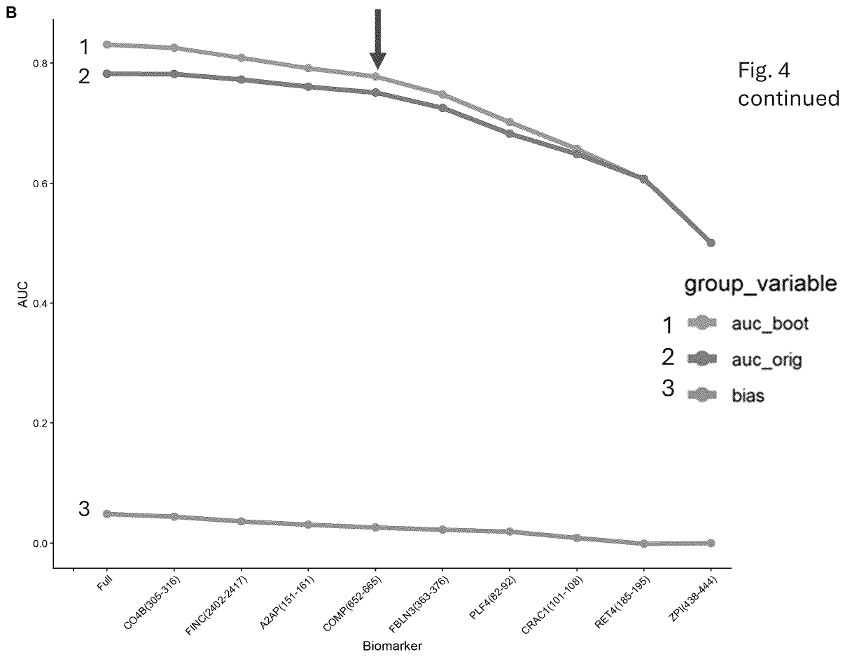

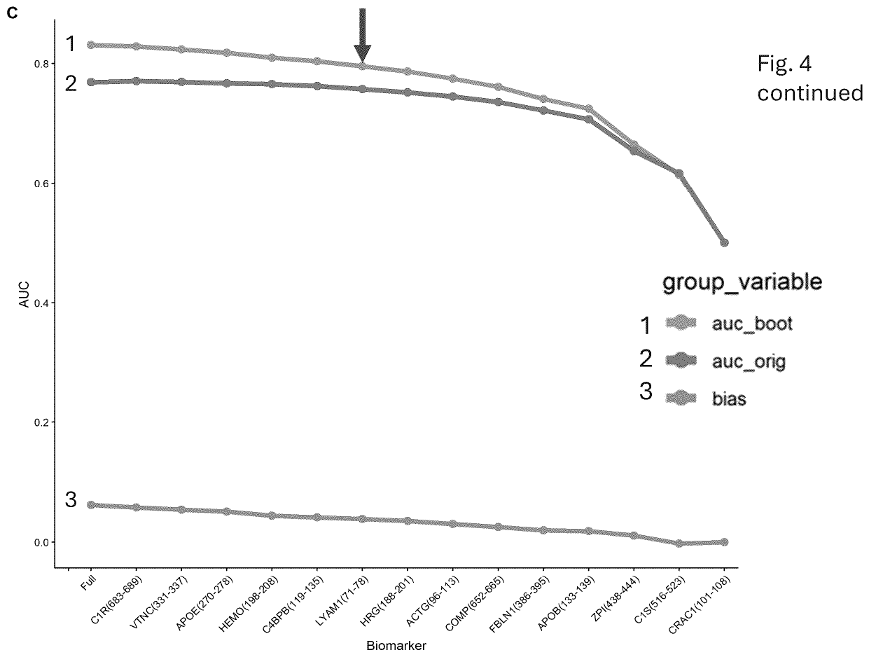

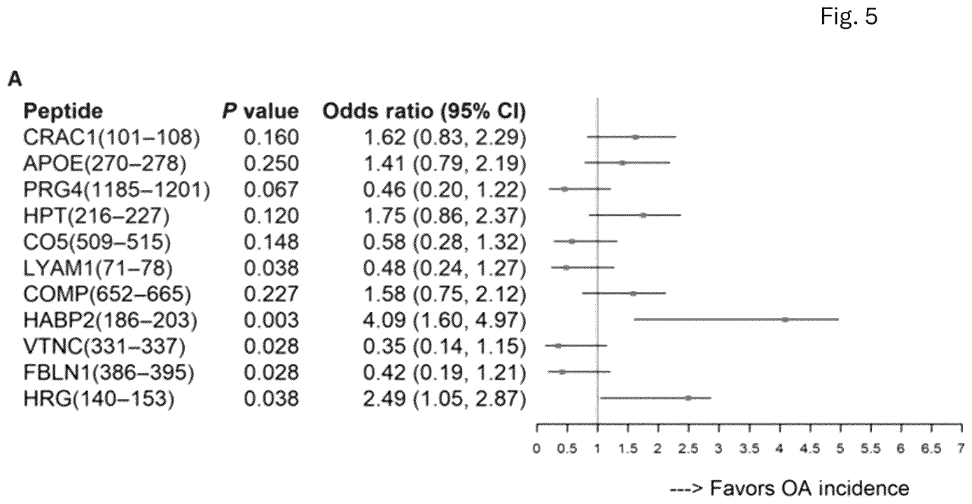

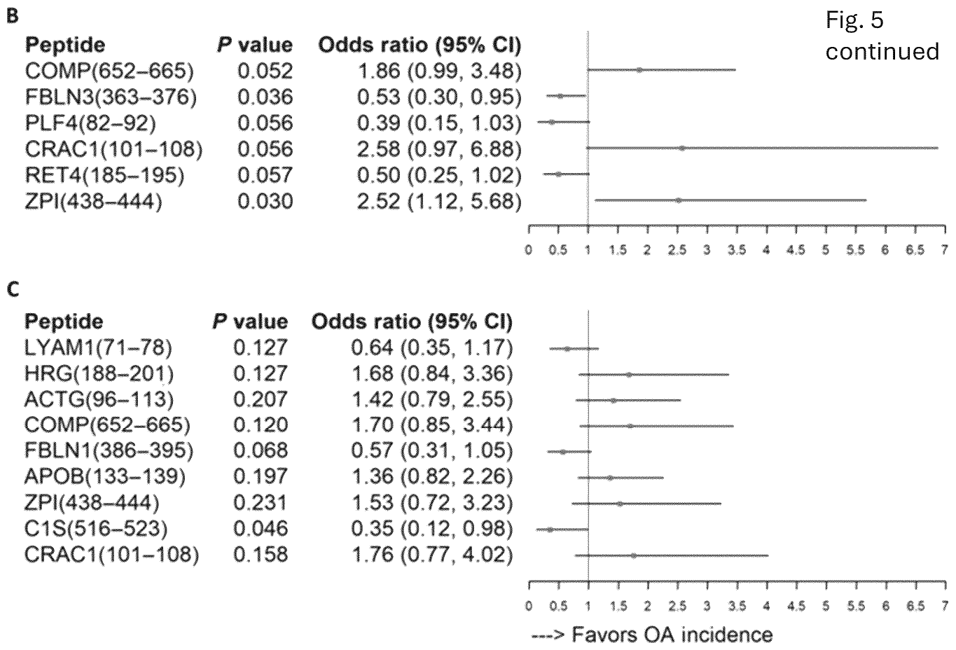

Full models, composed of 14, 9, and 14 biomarkers in the stable sets for y2, y6, and y2/6-TIC models, respectively, all yielded bootstrap area under the curve (AUC) of 0.83. After backward elimination to remove the peptides that contributed least to the overall AUCs (FIG. 4), there were 11, 6, and 9 biomarkers in the three datasets corresponding to y2, y6, and y2/6-TIC predictors, respectively, with AUCs of 0.76 to 0.77 (Table 2); these were termed the “essential set” biomarkers. CRAC1(101-108) and COMP(652-665) appeared in all three essential sets (FIG. 5). The y2 biomarkers of the primary analysis also predicted y5 RKOA (AUC 0.71, Table 2). Secondary analysis of y2 biomarkers (excluding the y5 n=41 incident cases and n=3 with missing RKOA status) and reselecting biomarkers with elastic net yielded six essential biomarkers achieving a similar AUC (0.77) as the primary y2 analysis using all cases (0.76) but requiring only half the number of predictors (6 versus 12 peptides); five of these essential biomarkers overlapped with the original y2 biomarker essential set (Table 2). Sensitivity analyses demonstrated no impact of the baseline Western Ontario and McMaster Universities Arthritis Index (WOMAC) pain on walking, baseline knee KL grade 1, or y2 radiographic hip OA (RHOA) on y2 biomarker predictivity of y10 RKOA (Table 2).

TABLE 2
Prediction of y 10 RKOA for all models by AUC (95% CIs) (n).

| | | Year 2 | | |
|---|---|---|---|---|
| | | biomarker | | |
| | Year 2 | predict y 10 | Year 6 | Biomarker |
| Essential | biomarker | RKOA | biomarker | y 2/6-TIC |
| biomarker | predict y 10 | secondary | predict y 10 | predict y 10 |
| models | RKOA | analysis* | RKOA | RKOA |
| Essential | 0.76 (0.70, 0.82) | 0.77 (0.69, 0.84) | 0.77 (0.67, 0.86) | 0.77 (0.69, 0.84) |
| biomarkers | (n = 196) | (n = 156) | (n = 197) | (n = 196) |
| only† | | | | |
| Essential | 0.76 (0.70, 0.82) | 0.76 (0.69, 0.83) | 0.76 (0.67, 0.85) | 0.76 (0.69, 0.84) |
| biomarkers and | (n = 196) | (n= 156) | (n = 197) | (n = 196) |
| demographics‡ | | | | |
| Essential | 0.83 (0.77, 0.89) | 0.77 (0.60, 0.84) | 0.78 (0.70, 0.85) | 0.83 (0.77, 0.89) |
| biomarkers, | (n = 164) | (n = 164) | (n = 165) | (n = 161) |
| demographics, | | | | |
| WOMAC A1, | | | | |
| and year 2 hip | | | | |
| OA | | | | |
| Essential | APOE(270-278) | APOE(270-278) | CRAC1(101-108) | ACTG(96-113) |
| biomarkers | CRAC1(101-108) | CRAC1(101-108) | COMP(652-665) | APOB(133-139) |
| | COMP(652-665) | CO5(509-515) | FBLN3(363-376) | CRAC1(101-108) |
| | CO5(509-515) | CD14(94-111) | PLF4(82-92) | COMP(652-665) |
| | FBLN1(386-395) | HABP2(186-203) | RET4(185-195) | C1S(516-523) |
| | HABP2(186-203) | HRG(140-153) | ZPI(438-444) | FBLN1(386-395) |
| | HPT(216-227) | | | HRG(188-201) |
| | HRG(140-153) | | | LYAM1(71-78) |
| | LYAM1(71-78) | | | ZPI(438-444) |
| | PRG4(1185-1201) | | | |
| | VTNC(331-337) | | | |
| | | | | Demographics, |
| | | | Year 2 | WOMAC A1, |
| | Demographics‡ | WOMAC A1 | hip OA | year 2 hip OA |
| | predict y 10 | predict y 10 | predict y 10 | predict y 10 |
| Clinical models | RKOA | RKOA | RKOA | RKOA |
| | 0.51 (0.42, 0.59) | 0.57 (0.51, 0.63) | 0.59 (0.55, 0.64) | 0.68 (0.60, 0.76) |
| | (n = 200) | (n = 170) | (n = 164) | (n = 164) |
| | | Predict y 10 | | |
| | | RKOA | Predict y 10 | Predict y 10 |
| Sensitivity | | excluding | RKOA | RKOA |
| analyses using | | baseline | excluding | excluding |
| year 2 essential | Predict y 5 | WOMAC A1 = | year 2 hip | baseline knee |
| biomarkers | RKOA | yes or missing | KL ≥ 2 | KL = 1 |
| | AUC | 0.80 (0.72, 0.87) | 0.78 (0.71, 0.85) | 0.79 (0.72, 0.86) |
| | 0.71 (0.63, 0.80) | (n = 134) | (n = 187) | (n = 179) |
| | (n = 157) | | | |

*Secondary analysis excluded year 5 prevalent and missing RKOA cases followed by elastic net reselection of the year 2 Essential biomarker set for predicting year 10 RKOA.
†The essential (final) biomarker sets consisted of selected biomarkers after stability screening and backward elimination.
‡Demographic variables are age and BMI at year 1 baseline.

The y6 biomarker only model yielded AUC [95% confidence interval (CI)] of 0.77 (0.67 to 0.86); this was similar to results using y2 and y2/6-TIC data but required only six peptides, the fewest number among all three main analyses (Table 2). The addition of age and BMI did not improve the discriminative capability (no change of AUCs) of any model; this was also consistent with the lack of discrimination by age and BMI only, AUC 0.51 (with AUC 0.50 reflecting no different than chance alone), which likely reflects the age and BMI matching between cases and controls. WOMAC pain and RHOA individually were slightly better discriminators of incident OA (AUCs, 0.57 to 0.59). Adding all clinical variables to essential biomarkers yielded a maximum AUC 0.83 (for y2 and y2/6-TIC) but with CIs overlapping those of models of essential biomarkers only (Table 2).

We also explored the combination of a few biomarkers, CRAC1(101-108) and COMP(652-665) with and without ZPI(438-444), the three biomarkers in all stable sets. These fewer biomarkers were still statistically significant predictors (lower AUC 95% CI >0.5) in yr2; the yr2 essential set AUC 0.76 fell within the upper CI of the AUC of the 3 peptide predictors of 0.80, so the 11 essential versus the 3 predictors were statistically indistinguishable. However, the two or three peptide predictors in yr6 were not statistically significant (lower CI of the AUC equaled or was below 0.5) so this combination was inferior to the full yr6 essential set of six peptides. The maximum AUCs (0.76) were achieved with yr2 or yr6 predictors, with baseline age, BMI, WOMAC A1, and RHOA (Table 5).

TABLE 5
Prediction of year 10 radiographic knee OA (y 10 RKOA) with
a subset of biomarkers in years 2 or 6 by AUC (95% CIs)

| | AUC (95% CIs) | |
|---|---|---|
| | | CRAC1(101-108), |
| | CRAC1(101-108), | COMP(652-665), |
| Biomarker Models | COMP(652-665) | ZPI(438-444) |
| Year 2 biomarkers only | 0.67 (0.56, 0.75) | 0.72 (0.61, 0.80) |
| Yr 2 Biomarkers + demo- | 0.70 (0.60, 0.78) | 0.73 (0.63, 0.81) |
| graphics | | |
| Yr 2 Biomarkers + WOMAC | 0.72 (0.61, 0.78) | 0.74 (0.63, 0.80) |
| A1, and year 2 Hip OA | | |
| Yr 2 Biomarkers + demo- | 0.76 (0.67, 0.82) | 0.76 (0.65, 0.81) |
| graphics, WOMAC A1, and | | |
| year 2 Hip OA | | |
| Year 6 biomarkers only | 0.63 (0.50, 0.72) | 0.63 (0.45, 0.70) |
| Yr 6 Biomarkers + demo- | 0.63 (0.42, 0.69) | 0.63 (0.39, 0.67) |
| graphics | | |
| Yr 6 Biomarkers + WOMAC | 0.73 (0.61, 0.81) | 0.74 (0.61, 0.81) |
| A1, and year 2 Hip OA | | |
| Yr 6 Biomarkers + demo | 0.76 (0.62, 0.84) | 0.76 (0.62, 0.82) |
| graphics, WOMAC A1, and | | |
| Hip OA year 2 | | |

Yr = year; Demographic variables were age and BMI in year 1. Additional variables were WOMAC A1 (pain on walking) in year 1 and radiographic hip OA (KL ≥ 2) in year 2.

Among the essential peptides, the highest odds ratio (ORs [95% CI]) for independently predicting incident RKOA in logistic regression models were achieved by y2 HABP2(186-203) (4.09 [1.60, 4.97]) and HRG(1.05-2.87) (2.49 [1.05, 2.87]) and y6 CRAC1(101-108) (2.58, [0.97,6.88]) and ZPI(438-444) (2.52 [1.12, 5.68]) (FIG. 5). For example, the OR 2.58 for CRAC1(101-108) means that when other biomarker concentrations remain unchanged, for every one-unit (equal to 1 SD) increase in CRAC1(101-108), we expect an 158% increased risk for incident RKOA in the subsequent 4 years. Some peptides were positively associated with incident RKOA [ACTG(96-113), APOB(133-139), APOE(270-278), COMP(652-665), CRAC1(101-108), HABP2(186-203), HPT(216-227), HRG(140-153), HRG(188-201), and ZPI(438-444); OR ranges: 1.36 to 4.09; 95% CIs provided in FIG. 5], while some were negatively associated [C1S(516-523), CO5(509-515), FBLN1(386-395), FBLN3(363-376), LYAM1(71-78), PLF4(82-92), PRG4(1185-1201), RET4(185-195), and VTNC(331-337); OR ranges, 0.35 to 0.64; 95% CIs provided in FIG. 5].

### Single-Cell Gene Expression and Pathway Analysis

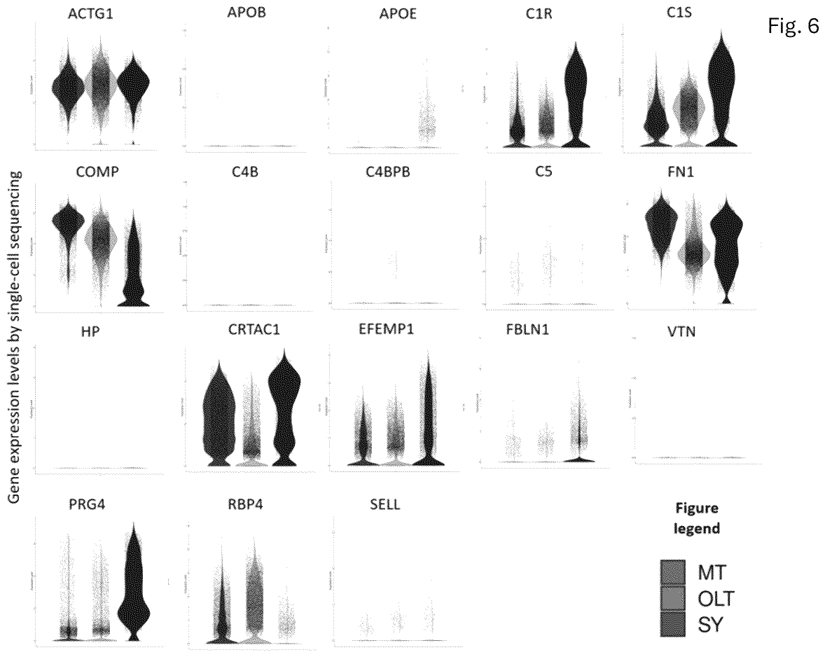

Single-cell gene expression corresponding to proteins in the stable sets was available from our prior analyses of knee OA cartilage and matched synovium (21). Genes corresponding to 18 of the 24 stable protein biomarkers were expressed in knee joint cartilage and/or synovium (FIG. 6) including ACTG1, C1R, C1S, COMP, CRTAC1, EFEMP1 (FBLN3), and FN1 (cartilage and synovium); RBP4 (RET4) (chondrocytes from lesioned and nonlesioned cartilage); and FBLN1 and PRG4 (both chondrocytes and most strongly synoviocytes). A2AP, HABP2, HEMO (HPX), HRG, PLF4, and ZPI were not found in the joint tissue transcriptome data, but were found in synovial fluid and thus may be found in the plasma.

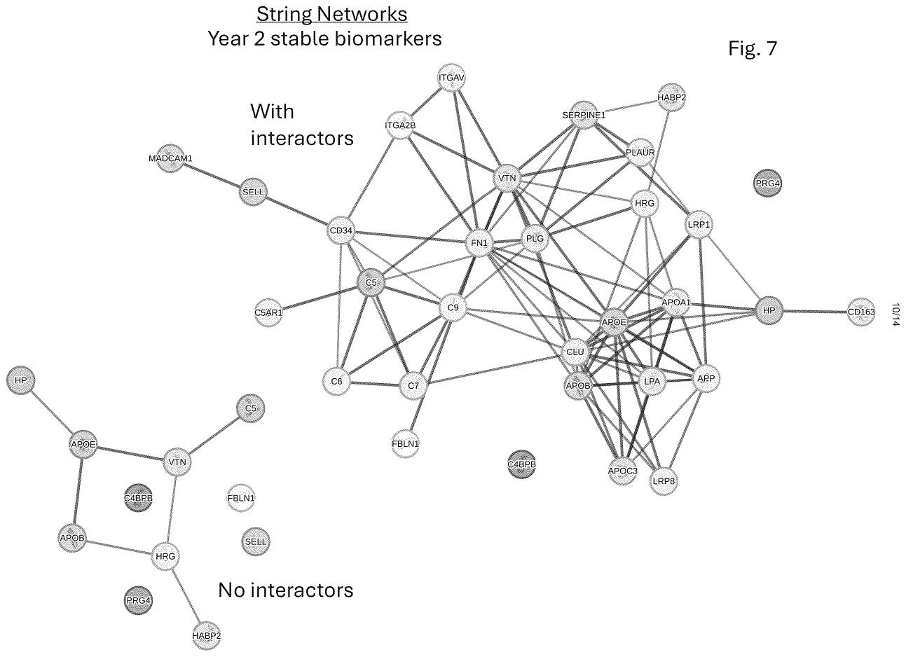

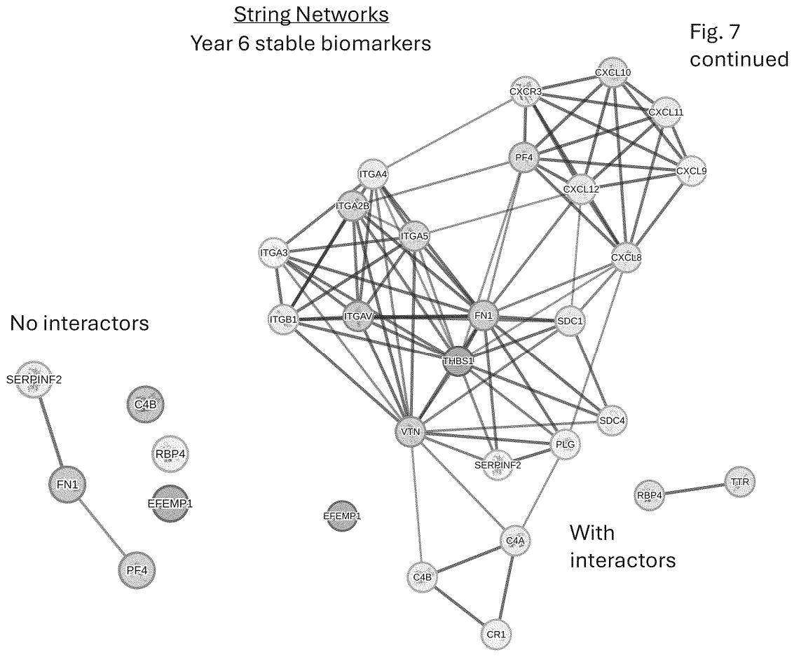

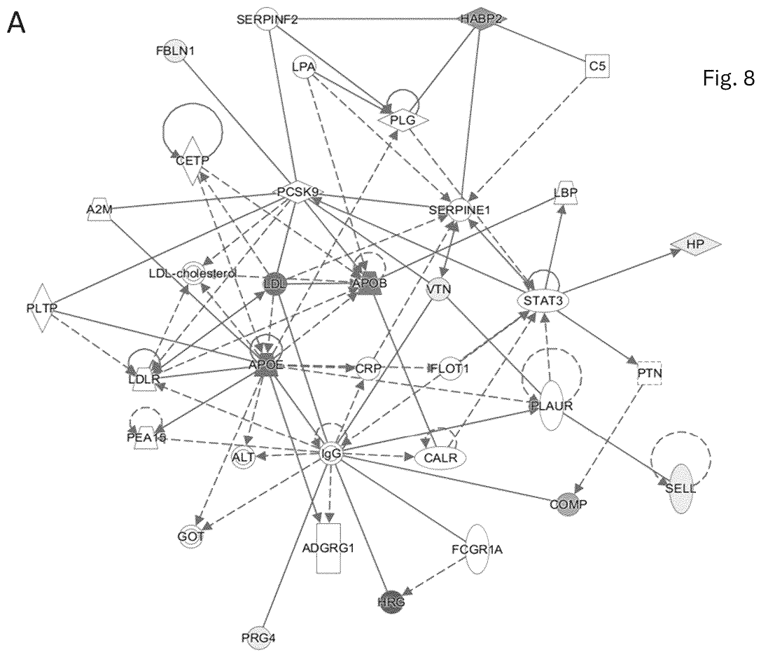

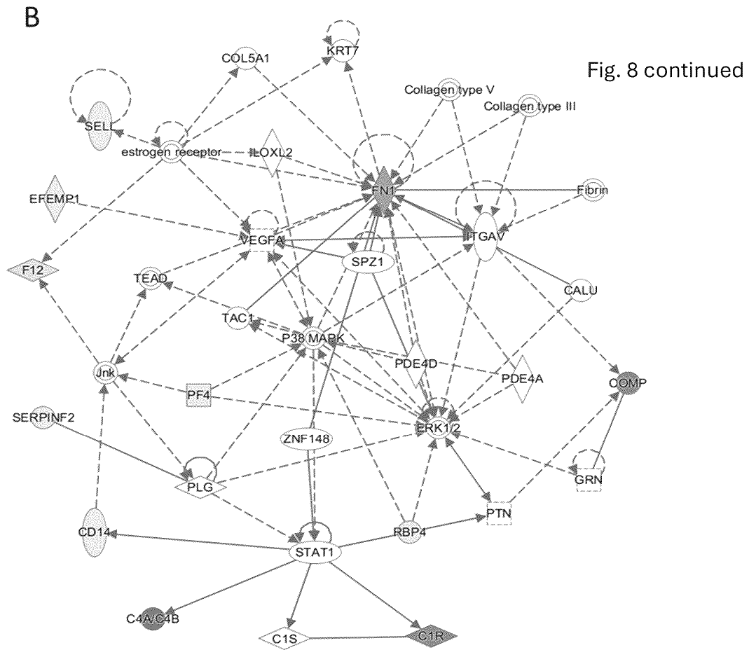

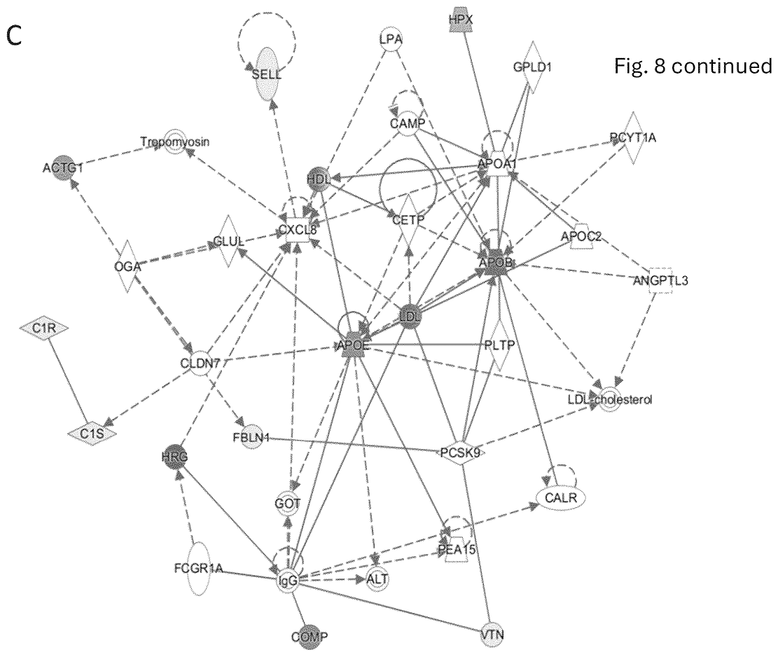

The stable biomarker sets in both yr2 and yr6 strongly implicate an unresolved acute phase response (APR) as the underlying pathophysiology in the years leading up to RKOA as they include proteins functioning in hemostasis with components related to coagulation, fibrin clot formation, and fibrinolysis [Platelet factor 4 (PLF4), Fibulin-1 (FBLN1), Histidine-rich glycoprotein (HRG), Protein Z-dependent protease inhibitor (ZPI), Fibronectin (FINC), Alpha-2-antiplasmin (A2AP), Hyaluronan-binding protein 2 (HABP2), Haptoglobin (HPT) and C4b-binding protein beta chain (C4BPB)] and proteins functioning in the complement cascade [Complement C1r subcomponent (C1R), Complement C1s subcomponent (C1S), Complement C4-B (CO4B), Complement C5 (CO5), Vitronectin (VTNC), Hemopexin (HEMO) and C4BPB]. Search Tool for the Retrieval of Interacting Genes/Proteins (STRING) network (FIG. 7) and Pathways Common analyses of the non-overlapping stable biomarkers identified blood coagulation, hemostasis regulation, negative wound healing, and regulation of the complement cascade for yr2 and hemostasis-related fibrin clot formation of a fibronectin (FINC)-centered network for yr6; extension of the yr2 network to include interactors also demonstrated the involvement of fibronectin. Notably, the fibronectin peptide selected as essential in this study resides in a C-terminal fibrin binding domain of fibronectin consistent with the pathway results. Consistent with these results, the top canonical pathways identified by Ingenuity Pathway Analysis (IPA network diagrams in FIG. 8) of the stable biomarker sets included APR signaling, LXR/RXR activation [recently shown to be the most up-regulated pathway in early OA based on synovial fluid proteomics (22)], FXR/RXR activation, the complement system, and clathrin-mediated endocytosis signaling. Low-density lipoprotein (LDL) receptor, LDL and LDL-cholesterol, were frequent connectors in the networks. Immunoglobulin G linked many nodes in the network, which may signify additional interactions with the immune system and consistent with the known association of OA with innate immunity.

### DISCUSSION

Using elastic net regression, we identified a small and parsimonious number of “essential” proteomic biomarkers that could discriminate, as much as 8 years prior, incident RKOA (based on change in KL grade) from non-RKOA in a subset of middle-aged women of the Chingford cohort. All models (y2, y6, and y2/6-TIC) based solely on the proteomic biomarker sets yielded cross-validated AUCs ≥0.76 for predicting incident RKOA. As few as six essential predictors (y2 secondary analysis and y6; AUCs, 0.77) predicted incident RKOA; this suggested that a select few biomarkers can be optimal for identifying the risk of incident RKOA occurring anytime up to 8 years later. The secondary analysis yielded an AUC 0.77, thereby demonstrating the ability of y2 biomarkers to also predict incident RKOA in the relatively distant future (i.e., RKOA incidence 3 or more years after the biomarker measures). Although it has been noted that the predictive value of risk factors often increases when the time to the outcome is smaller (23), in this study, the strength of prediction based on y2 and y6 essential biomarkers was equivalent, suggesting that it may be possible to predict incident RKOA even earlier than observed here. Individually, clinical characteristics (age, BMI, WOMAC pain on walking, and RHOA) were less predictive (AUCs, 0.51 to 0.59) than peptide biomarkers of incident y10 RKOA. Our study is relatively unique for selection of a cohort without enrichment for specific clinical or other risk factors, such as contralateral knee OA (24), self-reported knee injury, prior knee surgery, contralateral total knee replacement, baseline knee symptoms, or Heberden's nodes (25), as performed in other studies to increase the likelihood of incident RKOA.

Two peptides, COMP(652-665) and CRAC1(101-108), were selected in all stable and essential biomarker sets; ZPI(438-444) was also selected in all stable biomarker sets. These peptides alone, measured in yr2, significantly discriminated incident RKOA and were statistically indistinguishable from the full yr2 essential set predictors. COMP is a well-known OA-related biomarker indicating cartilage degeneration (26), OA disease status, incidence, and progression (15, 27-30). COMP fragments released from the cartilage extracellular matrix function as disease-associated molecular patterns (DAMPs) that activate joint inflammation (31). Our current results confirm that COMP is useful for identifying incident RKOA and agree with several prior studies that similarly identified COMP as a predictor of incident RKOA based on KL grade including the Chingford cohort (15), a meta-analysis of four cohorts (16), and the Johnston County Osteoarthritis Project (14), in contrast to one prior study in which it was reported to be a negative predictor of incident RKOA (18). CRAC1 functions as a cartilage degradation marker; it is a cartilage extracellular matrix protein, enriched in the upper layer of articular cartilage (32), that can distinguish chondrocytes from osteoblast-like cells and mesenchymal stem cells in culture (33, 34), predict severity and progression of RKOA (35), and risk of joint replacement (36). ZPI, protein Z-dependent protease inhibitor, is an anticoagulant protein of the serpin family that can rapidly inhibit membrane-associated coagulation factors Xa and Xia (37).

It is intriguing to note that despite the absence of overt injury in this cohort, nearly all of the stable set biomarkers predicting incident RKOA directly implicate an unresolved, perpetual APR (38), that leads to poor tissue repair and that has been identified previously as playing a pathogenic role in rheumatoid arthritis. The APR is made up of interacting and co-evolved processes (39) now known to be intimately involved in innate immunity, including hemostasis involving coagulation fibrin clot formation and fibrinolysis and the complement cascade. In addition, several of the biomarkers are known DAMPs that directly activate innate immunity, including fragments of fibronectin, PRG4 (lubricin), and COMP (31). COMP, fibronectin, and collagen II exposed on the articular cartilage surface bind lubricin (PRG4) responsible for boundary lubrication of the joint surfaces (40). Thus, in addition to activation of inflammatory pathways by fibronectin and COMP, the appearance of components of important extracellular matrix components—fibronectin, COMP, and PRG4—by y6 as essential and/or stable biomarkers, may signify the evolution to the loss of essential boundary lubrication of articular cartilage. To our knowledge, no literature yet reported three of the essential peptides identified in this study [CRAC1(101-108), LYAM1(71-78), and ZPI(438-444)], as predictors of incident RKOA, although we previously identified CRAC1 and ZPI as predictors of progression of established radiographic OA (19). LYAM1 (1-selectin) is a major regulator of leukocyte adhesion, migration, and signaling (41); serum concentrations are increased in patients with rheumatoid arthritis (42).

Although some of the proteins corresponding to this APR-related response were not found to be expressed in the joint tissue transcriptome data, including A2AP (SERPINF2), HABP2 (HABP2), HEMO (HPX), HRG (HRG), PLF4 (PF4), and ZPI (SERPINA10), they can all be found in synovial fluid because it is a dialysate of plasma that contains bioactive molecules from the circulation, in addition to those from joint tissues, that can mediate the OA process. Several of these APR-related biomarkers demonstrated previously to be in arthritic synovial fluid include HABP2 (43), HRG (early OA marker) (22), and ZPI (44), all positively associated with incident RKOA in our study, and PLF4 (45), negatively associated with incident RKOA in our study.

The ability of serum proteomic biomarkers to discriminate incident RKOA 4 to 8 years later in the Chingford cohort (AUCs, 0.76 to 0.77) was unexpectedly greater than discrimination of incident RKOA up to 1 to 5 years later achieved by magnetic resonance imaging features in other cohorts (AUC ranges of 0.59 to 0.68) (46-48). This could denote a molecular phase of OA that is best predicted by molecular measures. The first study to evaluate prediction of incident RKOA by different risk factors (clinical, genetic, and biochemical) identified baseline KL grade alone (AUCs, 0.70 to 0.84) as a predictor in two cohorts (23); the biochemical marker urinary CTXII alone was less predictive (AUCs, 0.60 to 0.61). Our results using proteomic predictors alone also compare favorably with results from a larger subsample (649 women) of the Chingford 1000 women cohort that achieved AUC 0.80 with baseline knee pain, KL grade, hip alpha angle, spine bone mineral density, and the presence of contralateral knee OA.

A prior proteomic study of the OAI cohort (AUC 0.79 in replication cohort) (25) yielded panels of peptides that alone could well discriminate incident RKOA. However, the cohort was enriched for incident OA risk factors; their clinical model (age, BMI, previous knee injury, and WOMAC pain) was also highly predictive (AUC, 0.75) of incident RKOA (25). Our cohort was not selected for incident RKOA risk factors, and cases and controls were matched for age and BMI. In our study, individual clinical variables yielded AUCs 0.51 to 0.59 in contrast to the OAI study in which the clinical model (baseline age, BMI, WOMAC pain, and RHOA) yielded AUC 0.68. In our study, the combination of essential biomarkers with clinical variables boosted the y2 and y2/6-TIC biomarker AUCs to 0.83. The imbalance due to lack of matching on WOMAC pain and RHOA status at baseline likely contributed to this result and suggest a potential benefit of matching on these additional variables in future incident RKOA studies and perhaps further accounting for OA status at other joint sites not accounted for in these analyses. In contrast to the OAI cohort in which ITIH1 alone was predictive of incident RKOA (25), in our study, five essential peptides, three positive [HABP2(186-203), HRG(140-153), and ZPI(438-444)] and two negative [C1S(516-523) and FBLN3(363-376)] predictors were statistically significant in multivariable models of incident RKOA. Moreover, as described above, statistically significant discrimination of incident RKOA could be achieved with as few as two to three yr2 predictors, COMP(652-665), CRAC1(101-108), and/or ZPI(438-444). It remains to be determined whether hip OA incidence and/or progression could be predicted by this panel of biomarkers. The limited literature on the subject of inflammation of hip OA would suggest that the inflammasome, a key component of innate immunity, plays a role (49). Thus, we would speculate that there may be a generalized preradiographic OA dysregulated innate immunological state leading to an unresolved APR contributing to development of incident hip as well as knee OA.

Our candidate biomarkers were derived from a panel developed to identify prognostic markers of clinically relevant knee OA (radiographic and pain) progression (19). Although risk factors for incident OA have been postulated to differ from those for OA progression (50), our results show that there is considerable overlap, based on proteomic biomarkers, in their molecular pathophysiology. By comparing the stable sets of both analyses at the protein level, we found that among all 24 protein biomarkers predicting incident RKOA, 14 (ACTG, APOE, C1R, C1S, CG4B, CO5, CRAC1, HABP2, HEMO, HRG, PLF4, PRG4, RET4, and ZPI) of them were also prognostic for RKOA progression with those in bold representing an APR (19), which means that more than half (58%) of the biomarkers predicting incident RKOA overlapped with biomarkers predicting RKOA progression and a majority of these reflect a dysregulated innate immune response and unresolved APR. Moreover, 10 of these (C1R, C1S, CG4B, CRAC1, HABP2, HEMO, HRG, PLF4, RET4, and ZPI), corresponding to 42%, overlapped with biomarkers that predicted clinically relevant OA (pain and radiographic) progression (19), again with nearly all reflecting dysregulated innate immunity with an unresolved APR. As Felson (51) has aptly pointed out, the distinction between incidence and progression is less clear-cut than is optimal, therefore defining these stages is challenging. These results are important as they suggest overlap in biomarker predictors, and therefore considerable similarity in the molecular pathophysiology of the progression to incident RKOA and the progression of established RKOA.

The ability of both y2 and y6 biomarkers to predict incident RKOA at y10 supports the existence of a prolonged molecular derangement of joint tissue before the onset of radiographic abnormality. This supports the OA iceberg concept (8) that an OA disease process begins long before a radiographic change and may even include an asymptomatic serological stage reflected only in molecular derangement discernible by tissue components released into the joint fluid or systemic circulation (6). Although we cannot conclude that the OA process is precisely the same throughout this time period (from preradiographic through to radiographic progression), we can confidently say that there are many similarities based on shared predictors (58%). The earliest OA continuum biomarkers (i.e., predicting incident RKOA more than 3 years in the future) may be APOE, CRAC1, CO5, HABP2, and HRG as they appeared in the y2 original and the y2 secondary analysis (excluding y5 RKOA cases). Further support for this is provided by a recent proteomic study showing that HRG was the strongest up-regulated synovial fluid protein comparing early OA (meniscal injury) to control (22).

There were several strengths of this study. Compared to traditional statistical approaches, such as univariate analysis followed by a multivariable logistic regression, machine learning approaches (elastic net is one) have greater predictive power because they are focused on making predictions as accurate as possible although this predictive power may come at the expense of other factors (overfitting, as one limitation) that can affect accuracy, while traditional statistical models are aimed at inferring relationships between variables (52). Moreover, elastic net is a method of choice for this study for several reasons: We could compare to prior results using the same method in an OA cohort (19), it deals with highly correlated variables, and it is appropriate for our situation wherein the number of predictors is larger or close to the sample size of the smallest class. The backward elimination process is also prediction oriented, aiming for fewer variables and the least AUC loss. Therefore, regarding prediction accuracy, it performs better than other predictive performance procedures such as conventional stepwise backward elimination based on AIC or P values or choosing unrelated biomarkers based on correlation coefficients. Since the starting point for backward selection was the stable sets, already a small set from feature selection, the computational cost was low. In addition, the biomarker panel evaluated in this study is well established as it has already been validated, in three independent cohorts, to contain biomarkers that predict progression of established RKOA (19, 32). Last, knee radiographic structural change and not symptoms was used as the outcome variable; so these biomarkers may have utility for selecting study participants for early OA clinical trials designed to develop and test disease-modifying OA drugs capable of benefiting joint structure.

There were several limitations of this study. First, the study was only conducted in women, however, none of the 17 essential predictors of incident OA in the Chingford cohort were significantly associated with sex in the FNIH cohort (19); moreover, none of these essential biomarkers were identified as sex-associated based on plasma proteomic analysis of 99 healthy adults that included 51 women (53). Second, sample size was relatively small. Therefore, we did not follow the regular training-testing procedure for AUC calculations as the separation would have further reduced the sample size. Instead, we used full samples to do the selection and fit the selected biomarkers to the same dataset, which will introduce bias and inflate the AUC. To adjust for this, we performed bootstrapping and deducted the bias of the bootstrapping estimate from the original AUC to yield a bias-corrected estimate. The sample size could also affect the biomarkers selected for the essential set. Third, there is no external validation for this study due to the difficulty of finding a similar longitudinal cohort with enough years of follow-up. Fourth, although imputation may have affected the peptide selection process, the overall missing rate was low (75% of the peptides had a missing rate less than 0.1% and half of the peptides had a missing rate of 0%); there were no missing data for y2 or y6 selected peptides and only three missing values for y2/6-TIC [related to FBLN3(363-376)]. Last, although the combination of individual clinical factors (age, BMI, WOMAC pain, and RHOA) yielded AUC 0.68, added to y2 molecular biomarkers the predictivity was boosted (to AUC 0.83); their combined contribution might prove significant in a larger sample. Further validation will be needed in independent and larger cohorts, preferably prospectively collected and including male participants and the combination of incident radiographic and symptomatic OA.

In summary, the present study identifies a small number of serum peptides that parsimoniously predicts incident RKOA up to 8 years before the appearance of radiographic abnormality. This is important as it provides more evidence for a preradiographic phase of disease. Our results also provide valuable information for understanding the molecular events of early disease that could inform strategies to develop disease-modifying drugs for preclinical OA. The value of our study is a panel that, in the absence of clinical factors indicative of high risk of knee OA, has the potential to discriminate individuals at risk of incident RKOA from those not at risk.

### Materials and Methods

### Study Design

The Chingford study is a population-based longitudinal cohort of 1003 women from a general practice in Chingford, North-East London, UK (54, 55). Since 1989, participants were assessed annually for osteoporosis and OA. A subset of women was identified for this case-control cohort based on available sera from y2 and y6 for biomarker analyses; they were matched on baseline age within 2 years and BMI within 3 kg/m2 (FIG. 1). There were two main outcome groups-incident case (n=100) and non-incident control (n=100) groups. All participants had KL grade ≤1 radiographic scores at baseline of both knees. Participants had no history at baseline of rheumatoid arthritis, gout, or previous joint injury (answered “no” to “Have you ever injured your knees enough to rest for a week?”). Incident RKOA was defined as KL grade >1 of either knee over the 10-year follow-up. Hip radiographic data were available for y2. Of the 100 incident RKOA cases, 16 had RHOA of either side (KL grade ≥2), and 2 had missing data; controls had no RHOA of either hip. Participants were assessed at baseline for knee pain based on WOMAC question A1, “How much pain have you had when walking on a flat surface?” All participants provided informed written consent. The study was approved by the Research Ethics Committee of the UK National Research Ethics Service Committee South Central Oxford A (reference numbers LREC R&WF96 and REC84131).

### Sample Preparation for LC-MRM Analysis

Nondepleted serum samples from y2 and y6 were analyzed blindly and in random order for the proteomic panel using a liquid chromatography-multiple reaction monitoring (LC-MRM) methodology that we previously described (19). Briefly, samples were processed in randomized groups of 60 samples, protein was quantified by a Bradford assay in duplicate, and three types of QC samples were prepared to provide a global reference standard, to assess differences in digestion efficiencies between sets, and to quantify intra/interplate variation of the targeted analytes. For sample digestions, 5 μl of each sample was diluted 1:40 with a 5.13% deoxycholate (DOC) surfactant such that the starting protein concentration was 1.25 μg/μl. Samples were reduced with 10 mM dithiolthreitol at 80° C. for 20 min and then alkylated with 20 mM iodoacetamide at room temperature for 40 min. Sequencing grade trypsin was added (1:10 enzyme to protein ratio to each sample) for 2 hours at 37° C. Samples were acidified with 1% trifluoroacetic acid and centrifuged to remove residual DOC before LC-MRM analysis. An exogenous stable isotope-labeled (SIL) peptide mixture (10 fmol) of 165 C13/N15-labeled peptides was added corresponding to 104 endogenous human proteins.

### Targeted LC-MRM Analysis

Quantitative LC-MRM was performed, blinded to the clinical data, on 1 g of protein digest spiked with SIL peptides corresponding to 165 peptides (104 proteins) using a nanoAcquity Ultra-performance liquid chromatography (UPLC) system (Waters Corp) coupled to a Waters Xevo TQ-XS triple quadrupole mass spectrometer via a nanoelectrospray ionization source. Briefly, the sample was first trapped on a Symmetry C18 300 mm by 180 mm trapping column [5 μl/min at 99.9/0.1 (v/v) water/acetonitrile], after which the analytical separation was performed with a 1.8-μm Acquity HSS T3 C18 75 μm by 150 mm column (Waters Corp) using a 55-min gradient of 5 to 40% acetonitrile with 0.1% formic acid at a flow rate of 400 nl/min and column temperature of 55° C. Data collection on the Xevo TQ-XS mass spectrometer was performed in a targeted mode following a method created in Skyline (MacCoss Laboratory, University of Washington). Data collected using Skyline were expressed as a ratio of peak areas of the light endogenous signal divided by the heavy SIL peptide.

### QC Procedures

Several QC sample types were processed. (i) To assess differences in digestion efficiencies between sets, a digestion QC standard was created by pooling 50 μl from the first set of 60 samples, which was then mixed and sub-aliquoted (at the intact protein level). All digestion QC standards were then frozen. Three random aliquots were thawed and digested with each sample block to assess differences in digestion efficiency between sets 1 and 10. The average intraplate digestion QC across 131 peptides was 10.1%; the average interplate digestion QC across 131 peptides was 22.9%. (ii) To assess intra/interplate variation of the targeted analytes, a sample pool QC (SPQC) sample was created from the initial pooled sample (used for digestion controls), digested using the same protocol as the patient samples, and then sub-aliquoted and frozen at −80° C. An aliquot was removed for each of the 10 plates and run periodically throughout the acquisition window with the same liquid chromatography-mass spectrometry method as the rest of the study. The average intraplate SPQC across 131 peptides was 9.9%; the average interplate SPQC across 131 peptides was 15.5%. Samples were reanalyzed when interplate digestion variation was >30% and/or interplate SPQC sample variation was >20%.

### Data Cleaning/QC

The quality of each MRM assay was determined by the dot product of the transition ratios in the heavy channel compared to the transition ratios in the light channel; the dot product is a strong indicator of interference in either channel and can identify potential targeted peptides below the limit of quantification. A total of 115 peptides with dot products ≥0.90 were retained for statistical analysis. After filtering by dot product, seven samples were excluded: four from y2 and three from y6; five of these were from the case group and two were from the control group; this resulted in a final analysis of 196, 197, and 196 samples for y2, y6, and y2/y6-TIC, respectively. Samples were excluded on the basis of: a peptide missing rate greater than 15%, insufficient amount of starting material, outliers in mean ratio of all peptides for this sample, and outliers detected by the first two principal components of a scatterplot. We also manually verified and/or adjusted peak retention times and integrations within Skyline. Any signals not of sufficient quality based on peak shape, interference, or low signal-to-noise ratio were deemed unreliable and values set to missing.

### Missing Data Imputation

There were no missing not at random (i.e., below limit of quantification) values; there were missing at random values verified by manually checking the Skyline data. The statistical analysis requires complete data; therefore, for retained peptides (<15% missing at random values), missing values were imputed by random forest (56) using the missRanger (57) R package. This nonparametric method of imputation is superior to other common imputation approaches for mass spectrometry-based missing at random values (58). To account for variability introduced by imputation, imputation was repeated 10 times to generate 10 complete datasets; Rubin's rule was used for pooling the 10 datasets (59). Because of the wide range of raw peptide values, imputed data were scaled to z-scores for further analysis.

### Statistical Analysis

### Study Design

The statistical analysis focused on testing each individual peptide represented by the ratio of the endogenous to exogenous SIL peptide; protein level summation of peptides was not performed. Using incident RKOA status of one knee at y10 as the outcome, we analyzed three sets of predictors: biomarkers measured at y2, y6, and TICs of biomarkers over y2/6-TIC derived from the AUC of biomarker measures across the specific time interval. After data cleaning, a total of 115 peptides were retained for statistical analysis in a final 196, 197, and 196 samples for y2, y6, and y2/y6-TIC, respectively. Peptides with missing rate <15% were imputed by random forest, and 10 imputed complete datasets were generated.

### Selection of Biomarker Set

We used the same variable selection strategy that we implemented in Zhou et al. (19) (FIG. 2). Considering the number of samples (~200) and peptides (115) retained for statistical analysis and correlation between peptides, we performed bootstrapped elastic net logistic regression (60) for variable selection, as we previously implemented (19). Briefly, the standard elastic net modeling is to optimize the two tuning parameters, α and λ, which was done by 10-fold cross-validation for a given a across a grid search of a from 0.1 to 0.9 with an increment of 0.1. Our elastic net approach incorporated a bootstrapping strategy; that is, we repeated the elastic net modeling of each a for 100 sets of bootstrap samples and computed the average misclassification rate. Optimal α was determined on the basis of the smallest average misclassification rate across different α values. Next, the optimal α and λ were used to perform elastic net variable selection in 100 sets of bootstrap samples, and the frequency of a peptide being selected (selection frequency) was computed. We ranked peptides based on their selection frequency and chose up to 16 top peptides. The decision to use 16 as the maximum number of peptides was based on the consideration of the average maximum number of variables permissible in the three models using a 5:1 rule [ratio of min(events, non-events) to number of candidate parameters in the model](61). For instance, as y6 had only 50 case participants without prevalent RKOA, the maximum number of variables chosen for backward selection for this model was 10; after filtering out any peptides in the same protein, nine peptides remained in the model. This procedure resulted in a final 14, 9, and 14 peptides subjected to backward elimination for y2, y6, and y2/y6-TIC models with one peptide per protein within each model. Across models, this resulted in two different peptide representatives for ApoB and HRG, while the remaining proteins had only one peptide representative. We denoted these selected peptides as the “stable biomarker” set; they totaled 26 peptides (24 proteins). However, the stable biomarkers could contain correlated peptides with interference in models due to collinearity. Therefore, using our previously published protocol (19), we used a backward elimination procedure as previously described (19) to further reduce the number of peptides from the stable sets. Here, we defined “AUC bias” based on the difference between areas under receiver operating characteristic curves (AUC) from the original samples and the average AUC from 1000 bootstrap samples. The initial AUC bias was based on all stable peptides in the model. A single stable peptide that contributed the least to AUC was eliminated subsequently in each backward selection step with AUC bias calculated for each step. The final model chosen was based on a rapid drop in AUC bias after peptide removal. The peptides left in the final pool were denoted the essential set of biomarkers consisting of 11, 6, and 9 biomarkers for y2, y6, and y2/6-TIC, respectively.

### Risk Models

Three multivariable logistic regressions were fit: (i) essential biomarker sets only; (ii) essential biomarker sets with demographic variables (age and BMI); and (iii) essential biomarker sets with all clinical variables (age, BMI, WOMAC A1 pain, and RHOA). We assessed the models' predictive performance using AUCs (62, 63); AUC ranges between 0.5 and 1, with higher values indicating better discrimination between individuals who are high risk and low risk. A total of 1000 bootstrap samples were used to obtain bias-corrected estimates and the confidence interval of the model AUC as well as model coefficients. The model fitting was applied independently to each imputed dataset with results combined by Rubin's rule (59). Because of development of cases of RKOA by y5, we also performed a secondary analysis excluding RKOA cases at y5 to test the predictivity of biomarkers in y2 for RKOA development in years 6 to 10; for this analysis, the biomarkers were selected using the same variable selection method. To address the group imbalance in clinical variables (WOMAC A1, baseline knee KL grades, and y2 RHOA), we performed four sensitivity analyses testing the predictivity of y2 biomarkers for (i) RKOA in y5, (ii) RKOA in y10 excluding those with baseline WOMAC pain on walking or missing WOMAC data, (iii) RKOA in y10 excluding those with baseline RKOA (KL=1) of either knee, (iv) RKOA in y10 excluding those with y2 RHOA (KL≥2).

### Pathway Analysis and Network Visualization

We explored protein networks and canonical pathways associated with the selected stable protein sets using STRING version 12.0, Pathway Commons (2019 update), and QIAGEN's IPA (IPA, QIAGEN Redwood City, qiagen.com/ingenuity). Network graphs were generated by STRING and IPA.

### REFERENCES

1. Quicke J. G., Conaghan P. G., Corp N., Peat G., Osteoarthritis year in review 2021: Epidemiology & therapy. Osteoarthr. Cartil. 30, 196-206 (2022).
2. Collaborators G., Global, regional, and national incidence, prevalence, and years lived with disability for 310 diseases and injuries, 1990-2015: A systematic analysis for the Global Burden of Disease Study 2015. Lancet 388, 1545-1602 (2016).
3. Mahmoudian A., Lohmander L. S., Jafari H., Luyten F. P., Towards classification criteria for early-stage knee osteoarthritis: A population-based study to enrich for progressors. Semin. Arthritis Rheum. 51, 285-291 (2021).
4. Mahmoudian A., Lohmander L. S., Mobasheri A., Englund M., Luyten F. P., Early-stage symptomatic osteoarthritis of the knee—time for action. Nat. Rev. Rheumatol. 17, 621-632 (2021).
5. Luyten F. P., Bierma-Zeinstra S., Dell'Accio F., Kraus V. B., Nakata K., Sekiya I., Arden N. K., Lohmander L. S., Toward classification criteria for early osteoarthritis of the knee. Semin. Arthritis Rheum. 47, 457-463 (2018).
6. V. Kraus, “Preclinical and Early Osteoarthritis” in Rheumatology E-Book, M. Hochberg, A. Silman, J. Smolen, M. Weinblatt, W. MH, E. Gravallese, Eds. (Elsevier, ed. 7th, 2019), chap. 187.
7. V. Kraus, T. Vincent, “Osteoarthritis” in Goldman-Cecil Medicine, L. Goldman, A. Schafer, Eds. (Elsevier, ed. 26th, 2020), vol. 2, chap. 246, pp. 1698-1703.
8. M. Hochberg, V. Kraus, S. Lohmander, A. Guermazi, F. Roemer, A. Mobasheri, “Osteoarthritis” in Clinical Innovation in Rheumatology, Past, Present, and Future, J. Liebowitz, P. Seo, Eds. (CRC Press, Taylor and Francis, 2022), chap. 5, pp. 47-63.
9. van Spil W. E., Degroot J., Lems W. F., Oostveen J. C., Lafeber F. P., Serum and urinary biochemical markers for knee and hip-osteoarthritis: A systematic review applying the consensus BIPED criteria. Osteoarthr. Cartil. 18, 605-612 (2010).
10. van Spil W. E., Szilagyi I. A., Osteoarthritis year in review 2019: Biomarkers (biochemical markers). Osteoarthr. Cartil. 28, 296-315 (2020).
11. Convill J. G., Tawy G. F., Freemont A. J., Biant L. C., Clinically relevant molecular biomarkers for use in human knee osteoarthritis: A systematic review. Cartilage 13, 1511s-1531s (2021).
12. Kraus V. B., Biomarkers as drug development tools: Discovery, validation, qualification and use. Nat. Rev. Rheumatol. 14, 354-362 (2018).
13. Golightly Y. M., Kraus S. W., Renner J. B., Villaveces A., Casteel C., Jordan J. M., Serum cartilage oligomeric matrix protein, hyalutonan, high-sensitivity C-reactive protein and keratan sulfate as predictors of incident radiographic knee osteoarthritis: Differences by chronic knee symptoms. Osteoarthr. Cartil. 18, S62-S63 (2010).
14. Golightly Y. M., Marshall S. W., Kraus V. B., Renner J. B., Villaveces A., Casteel C., Jordan J. M., Biomarkers of incident radiographic knee osteoarthritis: Do they vary by chronic knee symptoms? Arthritis Rheum. 63, 2276-2283 (2011).
15. Blumenfeld O., Williams F. M., Hart D. J., Spector T. D., Arden N., Livshits G., Association between cartilage and bone biomarkers and incidence of radiographic knee osteoarthritis (RKOA) in UK females: A prospective study. Osteoarthr. Cartil. 21, 923-929 (2013).
16. Valdes A. M., Meulenbelt I., Chassaing E., Arden N. K., Bierma-Zeinstra S., Hart D., Hofman A., Karsdal M., Kloppenburg M., Kroon H. M., Slagboom E. P., Spector T. D., Uitterlinden A. G., van Meurs J. B., Bay-Jensen A. C., Large scale meta-analysis of urinary C-terminal telopeptide, serum cartilage oligomeric protein and matrix metalloprotease degraded type II collagen and their role in prevalence, incidence and progression of osteoarthritis. Osteoarthr. Cartil. 22, 683-689 (2014).
17. Saberi Hosnijeh F., Siebuhr A. S., Uitterlinden A. G., Oei E. H., Hofman A., Karsdal M. A., Bierma-Zeinstra S. M., Bay-Jensen A. C., van Meurs J. B., Association between biomarkers of tissue inflammation and progression of osteoarthritis: Evidence from the Rotterdam study cohort. Arthritis Res. Ther. 18, 81 (2016).
18. Van Spil W. E., Welsing P. M., Bierma-Zeinstra S. M., Bijlsma J. W., Roorda L. D., Cats H. A., Lafeber F. P., The ability of systemic biochemical markers to reflect presence, incidence, and progression of early-stage radiographic knee and hip osteoarthritis: Data from CHECK. Osteoarthr. Cartil. 23, 1388-1397 (2015).
19. Zhou K., Li Y.-J., Soderblom E., Reed A., Jain V., Sun S., Moseley M., Kraus V., A ‘best-in-class’ systemic biomarker predictor of clinically relevant knee osteoarthritis structural and pain progression. Sci. Adv. 9, eabq5095 (2023).
20. Garriga C., Sanchez-Santos M. T., Judge A., Hart D., Spector T., Cooper C., Arden N. K., Predicting incident radiographic knee osteoarthritis in middle-aged women within four years: The importance of knee-level prognostic factors. Arthritis Care Res. 72, 88-97 (2020).
21. Chou C.-H., Jain V., Gibson J., Attarian D. E., Haraden C. A., Yohn C. B., Laberge R.-M., Gregory S., Kraus V. B., Synovial cell cross-talk with cartilage plays a major role in the pathogenesis of osteoarthritis. Sci. Rep. 10, 10868 (2020).
22. Ali N., Turkiewicz A., Hughes V., Folkesson E., Tjörnstand J., Neuman P., Önnerfjord P., Englund M., Proteomics profiling of human synovial fluid suggests increased protein interplay in early-osteoarthritis (OA) that is lost in late-stage OA. Mol. Cell. Proteomics 21, 100200 (2022).
23. Kerkhof H. J., Bierma-Zeinstra S. M., Arden N. K., Metrustry S., Castano-Betancourt M., Hart D. J., Hofman A., Rivadeneira F., Oei E. H., Spector T. D., Uitterlinden A. G., Janssens A. C., Valdes A. M., van Meurs J. B., Prediction model for knee osteoarthritis incidence, including clinical, genetic and biochemical risk factors. Ann. Rheum. Dis. 73, 2116-2121 (2014).
24. Bay-Jensen A. C., Bihlet A., Byrjalsen I., Andersen J. R., Riis B. J., Christiansen C., Michaelis M., Guehring H., Ladel C., Karsdal M. A., Serum C-reactive protein metabolite (CRPM) is associated with incidence of contralateral knee osteoarthritis. Sci. Rep. 11, 6583 (2021).
25. Lourido L., Balboa-Barreiro V., Ruiz-Romero C., Rego-Perez I., Camacho-Encina M., Paz-González R., Calamia V., Oreiro N., Nilsson P., Blanco F. J., A clinical model including protein biomarkers predicts radiographic knee osteoarthritis: A prospective study using data from the Osteoarthritis Initiative. Osteoarthr. Cartil. 29, 1147-1154 (2021).
26. Tseng S., Reddi A. H., Di Cesare P. E., Cartilage oligomeric matrix protein (COMP): A biomarker of arthritis. Biomark Insights 4, 33-44 (2009).
27. Clark A. G., Jordan J. M., Vilim V., Renner J. B., Dragomir A. D., Luta G., Kraus V. B., Serum cartilage oligomeric matrix protein reflects osteoarthritis presence and severity: The Johnston County Osteoarthritis Project. Arthritis Rheum. 42, 2356-2364 (1999).
28. Kluzek S., Bay-Jensen A. C., Judge A., Karsdal M. A., Shorthose M., Spector T., Hart D., Newton J. L., Arden N. K., Serum cartilage oligomeric matrix protein and development of radiographic and painful knee osteoarthritis. A community-based cohort of middle-aged women. Biomarkers 20, 557-564 (2015).
29. Zhang J., Meta-analysis of serum C-reactive protein and cartilage oligomeric matrix protein levels as biomarkers for clinical knee osteoarthritis. BMC Musculoskelet. Disord. 19, 22 (2018).
30. Vilim V., Olejarova M., Machacek S., Gatterova J., Kraus V., Pavelka K., Serum levels of cartilage oligomeric matrix protein (COMP) correlate with radiographic progression of knee osteoarthritis. Osteoarthr. Cartil. 10, 707-713 (2002).
31. Lambert C., Zappia J., Sanchez C., Florin A., Dubuc J. E., Henrotin Y., The damage-associated molecular patterns (DAMPs) as potential targets to treat osteoarthritis: Perspectives from a review of the literature. Front Med (Lausanne) 7, 607186 (2021).
32. Kraus V., Reed A., Soderblom E., Moseley M., Hsueh M.-F., Attur M., Samuels J., Abramson S., Li Y.-J., Serum proteomic panel validated for prediction of knee osteoarthritis progression. Osteoarthr. Cartil. Open 6, 100425 (2024). 33. Steck E., Benz K., Lorenz H., Loew M., Gress T., Richter W., Chondrocyte expressed protein-68 (CEP-68), a novel human marker gene for cultured chondrocytes. Biochem. J. 353, 169-174 (2001).
34. Steck E., Braun J., Pelttari K., Kadel S., Kalbacher H., Richter W., Chondrocyte secreted CRTAC1: A glycosylated extracellular matrix molecule of human articular cartilage. Matrix Biol. 26, 30-41 (2007).
35. Szilagyi I. A., Vallerga C. L., Boer C. G., Schiphof D., Ikram M. A., Bierma-Zeinstra S. M. A., van Meurs J. B. J., Plasma proteomics identifies CRTAC1 as a biomarker for osteoarthritis severity and progression. Rheumatology (Oxford) 62, 1286-1295 (2023).
36. Styrkarsdottir U., Lund S. H., Saevarsdottir S., Magnusson M. I., Gunnarsdottir K., Norddahl G. L., Frigge M. L., Ivarsdottir E. V., Bjornsdottir G., Holm H., Thorgeirsson G., Rafnar T., Jonsdottir I., Ingvarsson T., Jonsson H., Sulem P., Thorsteinsdottir U., Gudbjartsson D., Stefansson K., The CRTAC1 protein in plasma associates with osteoarthritis and predicts progression to joint replacements: A large-scale proteomics scan in Iceland. Arthritis Rheumatol. 73, 2025-2034 (2021).
37. Han X., Fiehler R., Broze G. J. Jr., Characterization of the protein Z-dependent protease inhibitor. Blood 96, 3049-3055 (2000).
38. Gibson B. H. Y., Duvernay M. T., Moore-Lotridge S. N., Flick M. J., Schoenecker J. G., Plasminogen activation in the musculoskeletal acute phase response: Injury, repair, and disease. Res. Pract. Thromb. Haemost. 4, 469-480 (2020).
39. Keragala C. B., Draxler D. F., McQuilten Z. K., Medcalf R. L., Haemostasis and innate immunity—a complementary relationship: A review of the intricate relationship between coagulation and complement pathways. Br. J. Haematol. 180, 782-798 (2018).
40. Flowers S. A., Zieba A., Ornros J., Jin C., Rolfson O., Bjorkman L. I., Eisler T., Kalamajski S., Kamali-Moghaddam M., Karlsson N. G., Lubricin binds cartilage proteins, cartilage oligomeric matrix protein, fibronectin and collagen II at the cartilage surface. Sci. Rep. 7, 13149 (2017).
41. Ivetic A., Hoskins Green H. L., Hart S. J., L-selectin: A major regulator of leukocyte adhesion, migration and signaling. Front. Immunol. 10, 1068 (2019).
42. Littler A. J., Buckley C. D., Wordsworth P., Collins I., Martinson J., Simmons D. L., A distinct profile of six soluble adhesion molecules (ICAM-1, ICAM-3, VCAM-1, E-selectin, L-selectin and P-selectin) in rheumatoid arthritis. Br. J. Rheumatol. 36, 164-169 (1997).
43. Bhattacharjee M., Balakrishnan L., Renuse S., Advani J., Goel R., Sathe G., Keshava Prasad T. S., Nair B., Jois R., Shankar S., Pandey A., Synovial fluid proteome in rheumatoid arthritis. Proteomics 13, 12 (2016).
44. Keller L., Fortier L., Lattermann C., Hunt E., Zhang S., Fu Q., Jacobs C., Complement system dysregulation in synovial fluid from patients with persistent inflammation following anterior cruciate ligament reconstruction surgery. J. Cartil. Jt. Preserv. 3, 100114 (2023).
45. Endresen G. K., Evidence for activation of platelets in the synovial fluid from patients with rheumatoid arthritis. Rheumatol. Int. 9, 19-24 (1989).
46. Tack A., Ambellan F., Zachow S., Towards novel osteoarthritis biomarkers: Multi-criteria evaluation of 46,996 segmented knee MRI data from the Osteoarthritis Initiative. PLOS ONE 16, e0258855 (2021).
47. Okada K., Yamaguchi S., Sato Y., Enomoto T., Ogawa Y., Ohtori S., Tahara M., Sasho T., Comparison of meniscal extrusion and osteophyte formation at the intercondylar notch as a predictive biomarker for incidence of knee osteoarthritis-Data from the Osteoarthritis Initiative. J. Orthop. Sci. 24, 121-127 (2019).
48. Hirvasniemi J., Runhaar J., van der Heijden R. A., Zokaeinikoo M., Yang M., Li X., Tan J., Rajamohan H. R., Zhou Y., Deniz C. M., Caliva F., Iriondo C., Lee J. J., Liu F., Martinez A. M., Namiri N., Pedoia V., Panfilov E., Bayramoglu N., Nguyen H. H., Nieminen M. T., Saarakkala S., Tiulpin A., Lin E., Li A., Li V., Dam E. B., Chaudhari A. S., Kijowski R., Bierma-Zeinstra S., Oei E. H. G., Klein S., The KNee OsteoArthritis Prediction (KNOAP2020) challenge: An image analysis challenge to predict incident symptomatic radiographic knee osteoarthritis from MRI and X-ray images. Osteoarthr. Cartil. 31, 115-125 (2023).
49. Yokota S., Shimizu T., Matsumae G., Ebata T., Alhasan H., Takahashi D., Terkawi M. A., Iwasaki N., Inflammasome activation in the hip synovium of rapidly destructive coxopathy patients and its relationship with the development of synovitis and bone loss. Am. J. Pathol. 192, 794-804 (2022).
50. Lohmander L. S., Felson D., Can we identify a ‘high risk’ patient profile to determine who will experience rapid progression of osteoarthritis? Osteoarthristis Cartilage 12, 49-52 (2004).
51. Felson D. T., Identifying different osteoarthritis phenotypes through epidemiology. Osteoarthr. Cartil. 18, 601-604 (2010). [DOI][PMC free article][PubMed][Google Scholar]
52. Rajula H. S. R., Verlato G., Manchia M., Antonucci N., Fanos V., Comparison of conventional statistical methods with machine learning in medicine: Diagnosis, drug development, and treatment. Medicina (Kaunas) 56, 455 (2020).
53. Tebani A., Gummesson A., Zhong W., Koistinen I. S., Lakshmikanth T., Olsson L. M., Boulund F., Neiman M., Stenlund H., Hellström C., Karlsson M. J., Arif M., Dodig-Crnković T., Mardinoglu A., Lee S., Zhang C., Chen Y., Olin A., Mikes J., Danielsson H., von Feilitzen K., Jansson P. A., Angerås O., Huss M., Kjellqvist S., Odeberg J., Edfors F., Tremaroli V., Forsström B., Schwenk J. M., Nilsson P., Moritz T., Backhed F., Engstrand L., Brodin P., Bergström G., Uhlen M., Fagerberg L., Integration of molecular profiles in a longitudinal wellness profiling cohort. Nat. Commun. 11, 4487 (2020).
54. Hart D., Mootoosamy I., Doyle D., Spector T., The relationship between osteoarthritis and osteoporosis in the general population: The Chingford study. Ann. Rheum. Dis. 53, 158-162 (1994).
55. Hart D. J., Doyle D. V., Spector T. D., Incidence and risk factors for radiographic knee osteoarthritis in middle-aged women: The Chingford Study. Arthritis Rheum. 42, 17-24 (1999).
56. Stekhoven D. J., Buhlmann P., MissForest-non-parametric missing value imputation for mixed-type data. Bioinformatics 28, 112-118 (2012).
57. Wright M., Ziegler A., ranger: A fast implementation of random forests for high dimensional data inC++ and R. J. Stat. Softw. 77, 1-17 (2017).
58. Wei R., Wang J., Su M., Jia E., Chen S., Chen T., Ni Y., Missing value imputation approach for mass spectrometry-based metabolomics data. Sci. Rep. 8, 663 (2018).
59. D. Rubin, Multiple Imputation for Nonresponse in Surveys (John Wiley & Sons, 1987).
60. Zou H., Hastie T., Regularization and variable selection via the elastic net. J R. Stat. Soc. Stat. Methodol. Series B 67, 301-320 (2005).
61. Vittinghoff E., McCulloch C. E., Relaxing the rule of ten events per variable in logistic and Cox regression. Am. J. Epidemiol. 165, 710-718 (2007).
62. Hanley J., McNeil B., The meaning and use of the area under a receiver operating characteristic (ROC) curve. Radiology 143, 29-36 (1982).
63. DeLong E., DeLong D., Clarke-Pearson D., Comparing the areas under two or more correlated receiver operating characteristic curves: A nonparametric approach. Biometrics 44, 837-845 (1988).

### EMBODIMENTS

1. A method comprising (a) obtaining a biological sample from a subject and (b) measuring expression of a biomarker in the biological sample, wherein the biomarker comprises at least a portion of at least two biomarkers selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14, and combinations thereof, and wherein the biological sample comprises synovial fluid, chondrocytes, synoviocytes, blood, serum or plasma.

2. The method of embodiment 1, wherein the biomarkers are selected from the group consisting of CRAC1, COMP, ZPI, ACTG, APOB, APOE, C1S, CO5, FBLN1, FBLN3, HPT, HRG, HABP2, PLF4, PRG4, RET4, LYAM1, VTNC, CD14, and combinations thereof.

3. The method of embodiment 1, wherein the biomarkers are selected from the group consisting of CRAC1, COMP, ZPI, ACTG, APOB, APOE, C1S, CO5, FBLN1, FBLN3, HRG, HABP2, PLF4, PRG4, RET4, LYAM1, CD14, and combinations thereof.

4. The method of embodiment 1, wherein the biomarkers are selected from the group consisting of CRAC1, COMP, ZPI, APOE, CO5, FBLN3, HRG, HABP2, PLF4, RET4, CD14, and combinations thereof.

5. The method of embodiment 1, wherein the biomarkers comprise CRAC1, COMP, and ZPI.

6. The method of embodiment 5, wherein the biomarkers further comprise FBLN3, PLF4 and RET4.

7. The method of embodiment 1, wherein the biomarkers comprise at least one of ACTG₍₉₆₋₁₁₃₎, A2AP₍₁₅₁₋₁₆₁₎, APOB₍₁₃₃₋₁₃₉₎, APOB₍₄₀₂₂₋₄₀₃₄₎, APOE₍₂₇₀₋₂₇₈₎, C4BPB₍₁₁₉₋₁₃₅₎, CRAC1₍₁₀₁₋₁₀₈₎, CRAC1₍₁₇₀₋₁₇₈₎, COMP₍₆₅₂₋₆₆₅₎, C1R₍₆₈₃₋₆₈₉₎, C1S₍₅₁₆₋₅₂₃₎, CO4B₍₃₀₅₋₃₁₆₎, CO5₍₅₀₉₋₅₁₅₎, FBLN1₍₃₈₆₋₃₉₅₎, FBLN3₍₃₆₃₋₃₇₆₎, FINC₍₂₄₀₂₋₂₄₁₇₎, HPT₍₂₁₆₋₂₂₇₎, HEMO₍₁₉₈₋₂₀₈₎, HRG₍₁₄₀₋₁₅₃₎, HRG₍₁₈₈₋₂₀₁₎, HABP2₍₁₈₆₋₂₀₃₎, PLF4₍₈₂₋₉₂₎, ZPI₍₄₃₈₋₄₄₄₎, PRG4₍₁₁₈₅₋₁₂₀₁₎, RET4₍₁₈₅₋₁₉₅₎, LYAM1₍₇₁₋₇₈₎, VTNC₍₃₃₁₋₃₃₇₎, CD14₍₉₄₋₁₁₁₎, and combinations thereof.

8. The method of embodiment 1, wherein the biomarkers comprise at least one of SEQ ID NO: 1-28, and sequences with at least 90% identity to any one of SEQ ID NO: 1-28.

9. The method of any one of the preceding embodiments, wherein the biological sample is a serum sample.

10. The method of any one of the preceding embodiments, wherein the measuring the expression comprises mass spectroscopy or an affinity reagent-based detection system.

11. The method of embodiment 10, wherein the affinity reagent-based detection system is an ELISA, LFA or Western blot system.

12. The method of any one of the preceding embodiments, wherein the subject is over 50 years old.

13. The method of any one of the preceding embodiments, wherein the subject is at risk for osteoarthritis.

14. The method of any one of the preceding embodiments, wherein the subject has had a knee injury.

15. The method of embodiment 14, wherein the knee injury comprises an injury to a knee ligament, a knee bone or knee cartilage.

16. The method of embodiment 15, wherein the ligament knee injury comprises an injury to at least one of the anterior cruciate ligament (ACL), posterior cruciate ligament (PCL), medial collateral ligament (MCL), and lateral collateral ligament (LCL).

17. The method of embodiment 15, wherein the knee injury comprises an injury to the meniscus, the tibia, the patella, or femur.

18. The method of any one of the preceding embodiments, further comprising administering a therapeutic agent to the subject with increased expression of any one of the biomarkers and/or genes encoding the biomarkers or combinations thereof.

19. The method of embodiment 18, wherein the therapeutic agent comprises an analgesic, a non-steroidal anti-inflammatory drugs (NSAIDs), steroid injections, surgery, an anti-metabolic syndrome drug, miRNA-based therapeutic or combinations thereof.

20. The method of any one of the preceding embodiments, further comprising determining a risk of the subject developing knee osteoarthritis based on the measured expression of the at least two biomarkers.

21. The method of embodiment 20, wherein determining the risk of the subject developing knee osteoarthritis comprises applying a machine learning algorithm to the measured expression of the at least two biomarkers.

22. The method of any one of the preceding embodiments, wherein the subject does not exhibit radiographic abnormalities of knee osteoarthritis at the time of obtaining the biological sample.

23. The method of any one of embodiments 20-22, further comprising developing a treatment plan for the subject based on the determination of risk of the subject developing osteoarthritis.

24. A method of identifying a subject at risk of developing osteoarthritis, the method comprising:
(a) obtaining a biological sample from the subject, wherein the biological sample comprises synovial fluid, chondrocytes, synoviocytes, blood, serum or plasma;
(b) measuring the expression of a biomarker in the biological sample, wherein the biomarker comprises at least a portion of at least one of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof,
(c) comparing the expression of the biomarker(s) in the biological sample in step (b) to an expression level of the biomarker in a control sample;
(d) identifying the subject at risk of developing osteoarthritis when the expression of the biomarker(s) is altered as compared to the control; and
(e) administering a treatment to the subject for osteoarthritis based on the risk identified in step (d).

25. A method for treating osteoarthritis in a subject comprising:
(a) obtaining a biological sample from a subject;
(b) measuring expression of a biomarker in the biological sample, wherein the biomarker comprises at least a portion of a protein selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof,
(c) comparing the expression of the biomarker to a control;
(d) classifying the subject for likelihood of generation of osteoarthritis; and
(e) administering osteoarthritis treatment to the subject wherein the level of the biomarker indicates the subject is likely to develop osteoarthritis.

26. The method of embodiment 24 or 25, wherein the biomarker comprises at least a portion of CRAC1, COMP, ZPI, ACTG, APOB, APOE, C1S, CO5, FBLN1, FBLN3, HPT, HRG, HABP2, PLF4, PRG4, RET4, LYAM1, VTNC, CD14 and combinations thereof.

27. The method of embodiment 24 or 25, wherein the biomarker comprises at least a portion of CRAC1, COMP, ZPI, ACTG, APOB, APOE, C1S, CO5, FBLN1, FBLN3, HRG, HABP2, PLF4, PRG4, RET4, LYAM1, CD14 and combinations thereof.

28. The method of embodiment 24 or 25, wherein the biomarker comprises at least a portion of CRAC1, COMP, ZPI, APOE, CO5, FBLN3, HRG, HABP2, PLF4, RET4, CD14 and combinations thereof.

29. The method of embodiment 24 or 25, wherein the biomarker comprises at least a portion of CRAC1, COMP, ZPI and combinations thereof.

30. The method of embodiment 24 or 25, wherein the biomarker comprises at least one of ACTG₍₉₆₋₁₁₃₎, A2AP₍₁₅₁₋₁₆₁₎, APOB₍₁₃₃₋₁₃₉₎, APOB₍₄₀₂₂₋₄₀₃₄₎, APOE₍₂₇₀₋₂₇₈₎, C4BPB₍₁₁₉₋₁₃₅₎, CRAC1₍₁₀₁₋₁₀₈₎, CRAC1₍₁₇₀₋₁₇₈₎, COMP₍₆₅₂₋₆₆₅₎, C1R₍₆₈₃₋₆₈₉₎, C1S₍₅₁₆₋₅₂₃₎, CO4B₍₃₀₅₋₃₁₆₎, CO5₍₅₀₉₋₅₁₅₎, FBLN1₍₃₈₆₋₃₉₅₎, FBLN3₍₃₆₃₋₃₇₆₎, FINC₍₂₄₀₂₋₂₄₁₇₎, HPT₍₂₁₆₋₂₂₇₎, HEMO₍₁₉₈₋₂₀₈₎, HRG₍₁₄₀₋₁₅₃₎, HRG₍₁₈₈₋₂₀₁₎, HABP2₍₁₈₆₋₂₀₃₎, PLF4₍₈₂₋₉₂₎, ZPI₍₄₃₈₋₄₄₄₎, PRG4₍₁₁₈₅₋₁₂₀₁₎, RET4₍₁₈₅₋₁₉₅₎, LYAM1₍₇₁₋₇₈₎, VTNC₍₃₃₁₋₃₃₇₎, CD14₍₉₄₋₁₁₁₎, and combinations thereof.

31. The method of embodiment 24 or 25, wherein the biomarker comprises at least one of SEQ ID NO: 1-28, and sequences with at least 90% identity to any one of SEQ ID NO: 1-28.

32. The method of any one of embodiments 24-31, wherein the biological sample is a serum sample.

33. The method of any one of embodiments 24-32, wherein the measuring the expression comprises mass spectroscopy or an affinity reagent-based detection system.

34. The method of embodiment 33, wherein the affinity reagent-based detection system is an ELISA, LFA or Western blot system.

35. The method of any one of embodiments 24-34, wherein the subject is over 50 years old.

36. The method of any one of embodiments 24-35, wherein the subject does not exhibit radiographic signs of osteoarthritis.

37. The method of any one of embodiments 24-36, wherein the subject has had a knee injury.

38. The method of embodiment 37, wherein the knee injury comprises an injury to a knee ligament, a knee bone or knee cartilage.

39. The method of embodiment 38, wherein the ligament knee injury comprises an injury to the anterior cruciate ligament (ACL), posterior cruciate ligament (PCL), medial collateral ligament (MCL), and lateral collateral ligament (LCL).

40. The method of embodiment 38, wherein the knee injury comprises an injury to the meniscus, the tibia, the patella, or femur.

41. The method of any one of embodiments 37-40, wherein the subject had surgery to repair the injury to the knee.

42. The method of any one of embodiments 24-41, wherein the control sample is from a subject non suspected of having osteoarthritis or a standardized reference sample.

43. The method of any one of embodiments 24-41, wherein the control sample is from the same subject at a different time.

44. The method of any one of embodiments 24-43, wherein the biological sample is obtained from a subject within one year of a surgery.

45. The method of any one of embodiments 24-44, wherein the treatment comprises an anti-inflammatory agent, an analgesic, a surgery, an anti-metabolic syndrome drug, miRNA-based therapeutic, lifestyle changes, targeted weight management, neuromuscular strengthening, injury prevention strategies, or combination thereof.

What is claimed:

1. A method comprising (a) obtaining a biological sample from a subject and (b) measuring expression of a biomarker in the biological sample, wherein the biomarker comprises at least a portion of at least two biomarkers selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14, and combinations thereof, and wherein the biological sample comprises synovial fluid, chondrocytes, synoviocytes, blood, serum or plasma.

2. The method of claim 1, wherein the biomarkers are selected from the group consisting of CRAC1, COMP, ZPI, ACTG, APOB, APOE, C1S, CO5, FBLN1, FBLN3, HPT, HRG, HABP2, PLF4, PRG4, RET4, LYAM1, VTNC, CD14, and combinations thereof.

3. The method of claim 1, wherein the biomarkers are selected from the group consisting of CRAC1, COMP, ZPI, ACTG, APOB, APOE, C1S, CO5, FBLN1, FBLN3, HRG, HABP2, PLF4, PRG4, RET4, LYAM1, CD14, and combinations thereof.

4. The method of claim 1, wherein the biomarkers are selected from the group consisting of CRAC1, COMP, ZPI, APOE, CO5, FBLN3, HRG, HABP2, PLF4, RET4, CD14, and combinations thereof.

5. The method of claim 1, wherein the biomarkers comprise CRAC1, COMP, and ZPI.

6. The method of claim 5, wherein the biomarkers further comprise FBLN3, PLF4 and RET4.

7. The method of claim 1, wherein the biomarkers comprise at least one of ACTG₍₉₆₋₁₁₃₎, A2AP₍₁₅₁₋₁₆₁₎, APOB₍₁₃₃₋₁₃₉₎, APOB₍₄₀₂₂₋₄₀₃₄₎, APOE₍₂₇₀₋₂₇₈₎, C4BPB₍₁₁₉₋₁₃₅₎, CRAC1₍₁₀₁₋₁₀₈₎, CRAC1₍₁₇₀₋₁₇₈₎, COMP₍₆₅₂₋₆₆₅₎, C1R₍₆₈₃₋₆₈₉₎, C1S₍₁₆₋₅₂₃₎, CO4B₍₃₀₅₋₃₁₆₎, CO5₍₅₀₉₋₅₁₅₎, FBLN1₍₃₈₆₋₃₉₅₎, FBLN3₍₃₆₃₋₃₇₆₎, FINC₍₂₄₀₂₋₂₄₁₇₎, HPT₍₂₁₆₋₂₂₇₎, HEMO₍₁₉₈₋₂₀₈₎, HRG₍₁₄₀₋₁₅₃₎, HRG₍₁₈₈₋₂₀₁₎, HABP2₍₁₈₆₋₂₀₃₎, PLF4₍₈₂₋₉₂₎, ZPI₍₄₃₈₋₄₄₄₎, PRG4₍₁₁₈₅₋₁₂₀₁₎, RET4₍₁₈₅₋₁₉₅₎, LYAM1₍₇₁₋₇₈₎, VTNC₍₃₃₁₋₃₃₇₎, CD14₍₉₄₋₁₁₁₎, and combinations thereof.

8. The method of claim 1, wherein the biomarkers comprise at least one of SEQ ID NO: 1-28, and sequences with at least 90% identity to any one of SEQ ID NO: 1-28.

9. The method of claim 1, wherein the measuring the expression comprises mass spectroscopy or an affinity reagent-based detection system.

10. The method of claim 1, wherein the subject is over 50 years old, is at risk for osteoarthritis or has had a knee injury.

11. The method of claim 10, wherein the knee injury comprises an injury to a knee ligament, a knee bone, meniscus, or knee cartilage.

12. The method of claim 11, wherein the knee injury comprises an injury to at least one of the anterior cruciate ligament (ACL), posterior cruciate ligament (PCL), medial collateral ligament (MCL), and lateral collateral ligament (LCL).

13. The method of claim 1, further comprising administering a therapeutic agent to the subject with altered expression of any one of the biomarkers and/or genes encoding the biomarkers or combinations thereof.

14. The method of claim 13, wherein the therapeutic agent comprises an analgesic, a non-steroidal anti-inflammatory drugs (NSAIDs), steroid injections, a surgery, an anti-metabolic syndrome drug, miRNA-based therapeutic or combinations thereof.

15. The method of claim 1, further comprising determining a risk of the subject developing knee osteoarthritis based on the measured expression of the at least two biomarkers.

16. The method of claim 15, wherein determining the risk of the subject developing knee osteoarthritis comprises applying a machine learning algorithm to the measured expression of the at least two biomarkers.

17. The method of claim 15, further comprising developing a treatment plan for the subject based on the determination of risk of the subject developing osteoarthritis.

18. The method of claim 17, wherein the treatment comprises an anti-inflammatory agent, an analgesic, a surgery, an anti-metabolic syndrome drug, miRNA-based therapeutic, lifestyle changes or combinations thereof.

19. A method of identifying a subject at risk of developing osteoarthritis, the method comprising:
(a) obtaining a biological sample from the subject, wherein the biological sample comprises synovial fluid, chondrocytes, synoviocytes, blood, serum or plasma;
(b) measuring the expression of a biomarker in the biological sample, wherein the biomarker comprises at least a portion of at least one of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof,
(c) comparing the expression of the biomarker(s) in the biological sample in step (b) to an expression level of the biomarker in a control sample;
(d) identifying the subject at risk of developing osteoarthritis when the expression of the biomarker(s) is altered as compared to the control; and
(e) administering a treatment to the subject for osteoarthritis based on the risk identified in step (d).

20. A method for treating osteoarthritis in a subject comprising:
(a) obtaining a biological sample from a subject;
(b) measuring expression of a biomarker in the biological sample, wherein the biomarker comprises at least a portion of a protein selected from the group consisting of CRAC1, COMP, ZPI, ACTG, A2AP, APOB, APOE, C4BPB, C1R, C1S, CO4B, CO5, FBLN1, FBLN3, FINC1, HPT, HEMO, HRG, HABP2, PLF4, PRG4, RET4, LYAM1, VTNC, CD14 and combinations thereof,
(c) comparing the expression of the biomarker to a control;
(d) classifying the subject for likelihood of generation of osteoarthritis; and
(e) administering osteoarthritis treatment to the subject wherein the level of the biomarker indicates the subject is likely to develop osteoarthritis.


SEQUENCE LISTING (28 sequences)

SEQ ID NO: 1 - protein, 18 aa, Homo sapiens
VAPEEHPVLLTEAPLNPK

SEQ ID NO: 2 - protein, 11 aa, Homo sapiens
LCQDLGPGAFR

SEQ ID NO: 3 - protein, 7 aa, Homo sapiens
LAIPEGK

SEQ ID NO: 4 - protein, 13 aa, Homo sapiens
WNFYYSPQSSPDK

SEQ ID NO: 5 - protein, 9 aa, Homo sapiens
LQAEAFQAR

SEQ ID NO: 6 - protein, 17 aa, Homo sapiens
SQCLEDHTWAPPFPICK

SEQ ID NO: 7 - protein, 8 aa, Homo sapiens
SSPYYALR

SEQ ID NO: 8 - protein, 9 aa, Homo sapiens
GVASLFAGR

SEQ ID NO: 9 - protein, 14 aa, Homo sapiens
NALWHTGDTESQVR

SEQ ID NO: 10 - protein, 7 aa, Homo sapiens
GYGFYTK

SEQ ID NO: 11 - protein, 8 aa, Homo sapiens
LLEVPEGR

SEQ ID NO: 12 - protein, 12 aa, Homo sapiens
LVNGQSHISLSK

SEQ ID NO: 13 - protein, 7 aa, Homo sapiens
IIHFGTR

SEQ ID NO: 14 - protein, 10 aa, Homo sapiens
TGYYFDGISR

SEQ ID NO: 15 - protein, 14 aa, Homo sapiens
NPCQDPYILTPENR

SEQ ID NO: 16 - protein, 16 aa, Homo sapiens
EYLGAICSCTCFGGQR

SEQ ID NO: 17 - protein, 12 aa, Homo sapiens
DIAPTLTLYVGK

SEQ ID NO: 18 - protein, 11 aa, Homo sapiens
YYCFQGNQFLR

SEQ ID NO: 19 - protein, 14 aa, Homo sapiens
DSPVLIDFFEDTER

SEQ ID NO: 20 - protein, 14 aa, Homo sapiens
GGEGTGYFVDFSVR

SEQ ID NO: 21 - protein, 18 aa, Homo sapiens
FCEIGSDDCYVGDGYSYR

SEQ ID NO: 22 - protein, 11 aa, Homo sapiens
ICLDLQAPLYK

SEQ ID NO: 23 - protein, 7 aa, Homo sapiens
VVNPTLL

SEQ ID NO: 24 - protein, 17 aa, Homo sapiens
ITEVWGIPSPIDTVFTR

SEQ ID NO: 25 - protein, 11 aa, Homo sapiens
LIVHNGYCDGR

SEQ ID NO: 26 - protein, 8 aa, Homo sapiens
AEIEYLEK

SEQ ID NO: 27 - protein, 7 aa, Homo sapiens
QPQFISR

SEQ ID NO: 28 - protein, 18 aa, Homo sapiens
LTVGAAQVPAQLLVGALR